(19) United States
(12) Patent Application Publication
(10) Pub. No.: US 2019/0346456 A1
(43) Pub. Date: Nov. 14, 2019

(54) HIGH THROUGHPUT ANTIBODY VARIANT SCREENING METHOD

(71) Applicant: Just Biotherapeutics, Inc., Seattle, WA (US)

(72) Inventors: Rutilio H. Clark, Bainbridge Island, WA (US); Richard S. Rogers, Seattle, WA (US)

(73) Assignee: Just Therapeutics, Inc., Seattle, WA (US)

(21) Appl. No.: 16/409,818

(22) Filed: May 12, 2019

Related U.S. Application Data
(60) Provisional application No. 62/671,245, filed on May 14, 2018.

(51) Int. Cl. G01N 33/68; C07K 1/22; C07K 16/06; G01N 30/80; G01N 30/72

(52) U.S. Cl. CPC G01N 33/6854; G01N 33/6848; C07K 1/22; C07K 2317/14; G01N 30/80; G01N 30/7233; C07K 16/065

(57) ABSTRACT

Disclosed is a high throughput method for selecting an antibody variant amino acid sequence of interest from a plurality of antibody variant amino acid sequences. The method is particularly useful in the engineering of improved antibodies.

### CROSS-REFERENCES TO RELATED APPLICATIONS

This non-provisional patent application claims priority from U.S. Provisional Application Ser. No. 62/671,245, filed May 14, 2018, which is incorporated by reference in its entirety.

### SEQUENCE LISTING

The instant application contains a Sequence Listing which has been filed electronically in ASCII format and is hereby incorporated by reference in its entirety. Said ASCII copy, created on May 8, 2019, is named JUST0581US_SL.txt and is 733 bytes in size.

### BACKGROUND OF THE INVENTION

### 1. Field of the Invention

This invention relates to protein engineering and high throughput screening of variant recombinant antibodies.

### 2. Discussion of the Related Art

Antibodies are biologically and commercially significant polypeptides that bind with great specificity and affinity to a particular target molecule or antigen. The clinical value of certain antibodies as therapeutic molecules has long been recognized. However, antibodies that otherwise can be useful therapeutic molecules can also exhibit many undesirable properties disallowing for easy manufacture, storage and therapeutic delivery. (See, e.g., Daugherty, A. L. et al., Formulation and delivery issues for monoclonal antibody therapeutics, Advanced Drug Delivery Reviews 58(5-6):686-706 (2006); Vázquez-Rey, M., & Lang, D. a. (2011). Aggregates in monoclonal antibody manufacturing processes. Biotechnology and Bioengineering, 108(7), 1494-1508 (2011)).

Methods have been employed to address the need to improve detrimental antibody biophysical properties, including methods incorporating computational tools. (See, e.g., Clark, R. H. et al., Remediating agitation-induced antibody aggregation by eradicating exposed hydrophobic motifs, mAbs 6(6); 1540-1550 (2014); Kuroda, D. et al., Computer-aided antibody design, Protein Engineering, Design & Selection (PEDS) 25(10):507-21 (2012); Nichols, P. et al., Rational design of viscosity reducing mutants of a monoclonal antibody: Hydrophobic versus electrostatic inter-molecular interactions, mAbs 7(1):212-230 (2015); Talluri, S. Advances in engineering of proteins for thermal stability, International Journal of Advanced Biotechnology and Research 1:190-200 (2011); van der Kant, R. et al., Prediction and Reduction of the Aggregation of Monoclonal Antibodies, Journal of Molecular Biology 429(8):1244-1261 (2017); Voynov, V. et al., Predictive tools for stabilization of therapeutic proteins, mAbs 1(6):580-582 (2009)).

A challenging hurdle in the engineering and assaying process is interrogating a large design space addressing the potential liabilities to arrive at the top viable therapeutic candidates. (See, e.g., Igawa, T. et al., Engineering the variable region of therapeutic IgG antibodies, mAbs 3(3):243-252 (2011)). Generating thousands of antibodies individually and testing them on a high-throughput antibody-ligand interaction instrument can still be very laborious.

Accordingly, there is a need for a high throughput method whereby a large number of designed antibody variants are recombinantly produced simultaneously and are tested for binding to the target in a bulk fashion. This the present invention provides.

### SUMMARY OF THE INVENTION

The present invention relates to a high throughput method for selecting an antibody variant amino acid sequence of interest from a plurality of antibody variant amino acid sequences. It is a benefit of the inventive method that many different possible sequence variants can be generated recombinantly in relatively small quantities in a mixture for preliminary screening and selection of the most promising variants for further analysis, without generating material for each of the possible variants individually. The inventive high throughput method involves the steps of

(a) predetermining a plurality of variant amino acid sequences and the corresponding molecular weight of each member of the plurality of variant amino acid sequences, wherein the variant amino acid sequences are variants of a preselected reference antibody (e.g., a parental antibody reference sequence), wherein the reference antibody specifically binds to a target ligand of interest;

(b) cloning a plurality of nucleic acid sequences, each encoding a member of the plurality of variant amino acid sequences, to generate a mixed pool of nucleic acids capable of transfecting a mammalian cell;

(c) transfecting a plurality of mammalian cells with the mixed pool of nucleic acids from step (b);

(d) culturing the transfected mammalian cells under physiological conditions allowing the cells to express recombinant antibodies;

(e) harvesting the recombinant antibodies present in the culture in step (d) into a cell-free supernatant fraction and purifying the cell-free supernatant fraction by affinity chromatography to obtain a mixed pool of IgG molecules;

(f) loading the mixed pool of IgG molecules from step (e) onto an affinity chromatography matrix, wherein the target ligand of interest is covalently conjugated to the affinity chromatography matrix;

(g) eluting the IgG molecules from the affinity chromatography matrix under increasingly stringent buffer conditions and collecting a plurality of eluant fractions; and

(h) detecting the molecular weights of the IgG molecules present in each eluant fraction by mass spectrometry. Then because the calculated molecular weights of each of the predetermined variant amino acid sequences are known, one or more antibody variants of interest from the eluant fraction obtained under the highest stringency buffer conditions in step (g) can be identified by its corresponding molecular weight and can be selected from the plurality of variant amino acid sequences for further analysis.

The foregoing summary is not intended to define every aspect of the invention, and additional aspects are described in other sections, such as the Detailed Description of Embodiments. The entire document is intended to be related as a unified disclosure, and it should be understood that all combinations of features described herein are contemplated, even if the combination of features are not found together in the same sentence, or paragraph, or section of this document.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Also, aspects described as a genus or selecting a member of a genus, should be understood to embrace combinations of two or more members of the genus. Although the applicant(s) invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1 shows a schematic representation of an embodiment of the inventive method from expression of DNA variant pool to comparison of calculated antibody masses with empirical antibody masses.

FIG. 2 shows a schematic representation of an embodiment of cloning and recombinant antibody variant expression. “LC-x” and “HC-x” indicate light and heavy chain variant pools, respectively.

FIG. 3A-C show representative size exclusion chromatography (SEC) of a mixed pool of IgG molecules obtained from purifying a cell-free supernatant fraction by Protein A affinity chromatography. FIG. 3A shows SEC of the mAb_A parent antibody from a Protein A-purified pool. FIG. 3B shows SEC of the mAb_A variant HC pool and parental LC co-expression from a Protein A-purified pool. FIG. 3C shows SEC of the of the mAb_A variant LC pool and parental HC co-expression from a Protein A-purified pool.

FIG. 4 shows a schematic crystal structure of the mAb_A Fab—ligand complex. of the mAb_A Fab—ligand complex. Ligand is rendered in light grey ribbon whereas mAb_A (here designated “Fab_A”) is rendered in black ribbon. The dash line indicates missing electron density on the C-terminal end of ligand where the poly-histidine tag (grey circle) was attached. The CDR (complementary determining regions) molecular surface rendered as dots indicates the interaction surface with the target ligand. The interaction surface is defined as all atoms of the CDR within 4.5 Ängstroms of any ligand atom. Similarly, the solid molecular surface rendered on the target ligand indicates the interaction surface with the CDRs of Fab_A. The surfaces in such models can be colored to represent potential binding interactions between the surfaces such as lipophilicity or electrostatics. The rendered potential binding interactions provides information for mAb_A engineering.

FIG. 5 shows a deconvoluted spectrum from the deglycosylated mAb_A HC variant pool. Peak “abundance” corresponds to the abundance of species in a particular peak as a percentage the highest peak.

FIG. 6 shows a representative comparison of empirical antibody mass data with the calculated antibody masses of every designed HC variant of mAb_A; only the detected masses within the designated mass range that were present in the two fractions 4 and 6 are shown. Bars represent the calculated masses where relative abundance is the number of antibody variants with that calculated mass. The circle marker represents the empirical masses from fraction 4 whereas the triangle marker represents empirical masses from fraction 6. The relative abundance is the normalized signal for each deconvoluted mass found empirically. The dotted lines are merely visual aids for mass alignment.

FIG. 7 shows a representative Sanger sequencing chromatogram. The data show a portion (SEQ ID NO:1) of the VH region of mAb_A, where mixed bases were confirmed at the intended positions. IUPAC nucleotide codes were used where “W” is either an “A” or “T,” and “S” is either a “G” or “C.” FIG. 7 also discloses SEQ ID NO: 2.

### DETAILED DESCRIPTION OF EMBODIMENTS

The section headings used herein are for organizational purposes only and are not to be construed as limiting the subject matter described.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Thus, as used in this specification and the appended claims, the singular forms “a”, “an” and “the” include plural referents unless the context clearly indicates otherwise. For example, reference to “a protein” includes a plurality of proteins; reference to “a cell” includes populations of a plurality of cells.

The high throughput method for selecting an antibody variant amino acid sequence of interest from a plurality of antibody variant amino acid sequences includes the step of predetermining a plurality of variant amino acid sequences and the corresponding molecular weight of each member of the plurality of variant amino acid sequences, wherein the variant amino acid sequences are variants of a preselected parent antibody, wherein the parent antibody specifically binds to a target ligand of interest. The term “predetermined” or “predetermining” means that the variant amino acid sequences are chosen, elected, or selected, in advance of the cloning step, and that the predicted molecular weight of each variant amino acid sequence to be cloned and expressed is calculated and knowable. Methods for predetermining antibody variant amino acid sequences can include computational tools (e.g., PASTA, TANGO, and AGGRESCAN software platforms, e.g., for aggregation avoidance engineering) or other methods for rational design of antibodies to enhance their physiochemical properties, bioactivity, stability, ease of manufacture, storage and/or therapeutic delivery to a clinical patient.

Useful computational tools for predetermining the variant amino acid sequences also include Molecular Operating Environment, Discovery Studio, Rosetta, Biologics Suite, Triton, and Genedata Biologics. Other useful methods can include “germlining” for improvement of recombinant expression, affinity maturation and random CDR mutations, covariance analysis for enhanced stability, manufacturability, and/or expression, or by simple substitutions avoiding known potentials of chemical modification. In predetermining the variant amino acid sequences, the effects on stability and manufacturability of certain chemical modifications in the CDRs and/or the frameworks of the Fv should be considered. Possible effects of modifying particular amino acid residues that should be taken into account include, but are not limited to, isomerization, deamidation, N-link glycosylation, methionine oxidation, and/or cysteinylation. In silico molecular modeling and/or crystal and/or NMR structures of the preselected parental antibody (or other preselected reference sequence modified from the parental antibody) can be consulted for useful information in predetermining the variant amino acid sequences to be cloned. (See, also, e.g., Clark, R. H. et al., Remediating agitation-induced antibody aggregation by eradicating exposed hydrophobic motifs, mAbs 6(6); 1540-1550 (2014); Kuroda, D. et al., Computer-aided antibody design, Protein Engineering, Design & Selection (PEDS) 25(10):507-21 (2012); Nichols, P. et al., Rational design of viscosity reducing mutants of a monoclonal antibody: Hydrophobic versus electrostatic inter-molecular interactions, mAbs 7(1):212-230 (2015); Talluri, S. Advances in engineering of proteins for thermal stability, International Journal of Advanced Biotechnology and Research 1:190-200 (2011); van der Kant, R. et al., Prediction and Reduction of the Aggregation of Monoclonal Antibodies, Journal of Molecular Biology 429(8):1244-1261 (2017); Voynov, V. et al., Predictive tools for stabilization of therapeutic proteins, mAbs 1(6):580-582 (2009)).

A “stable” formulation is one in which the protein therein, e.g., an antibody, essentially retains its physical stability and/or chemical stability and/or biological activity upon processing (e.g., ultrafiltration, diafiltration, other filtering steps, vial filling), transportation, and/or storage of the antibody drug substance and/or drug product. Together, the physical, chemical and biological stability of the protein in a formulation embody the “stability” of the protein formulation, which is specific to the conditions under which the formulated drug product (DP) is stored. For instance, a drug product stored at subzero temperatures would be expected to have no significant change in either chemical, physical or biological activity while a drug product stored at 40° C. would be expected to have changes in its physical, chemical and biological activity with the degree of change dependent on the time of storage for the drug substance or drug product. The configuration of the protein formulation can also influence the rate of change. For instance, aggregate formation is highly influenced by protein concentration with higher rates of aggregation observed with higher protein concentration. Excipients are also known to affect stability of the drug product with, for example, addition of salt increasing the rate of aggregation for some proteins while other excipients such as sucrose are known to decrease the rate of aggregation during storage. Instability is also greatly influenced by pH giving rise to both higher and lower rates of degradation depending on the type of modification and pH dependence.

Various analytical techniques for measuring protein stability are available in the art and are reviewed, e.g., in Wang, W. (1999), Instability, stabilization and formulation of liquid protein pharmaceuticals, Int J Pharm 185:129-188. Stability can be measured at a selected temperature for a selected time period. For rapid screening, for example, the formulation may be kept at 40° C. for 2 weeks to 1 month, at which time stability is measured. Where the formulation is to be stored at 2-8° C., generally the formulation should be stable at 30° C. for at least 1 month, or 40° C. for at least a week, and/or stable at 2-8° C. for at least two years.

A protein “retains its physical stability” in a formulation if it shows minimal signs of changes to the secondary and/or tertiary structure (i.e., intrinsic structure), or aggregation, and/or precipitation and/or denaturation upon visual examination of color and/or clarity, or as measured by UV light scattering or by size exclusion chromatography, or other suitable methods. Physical instability of a protein, i.e., loss of physical stability, can be caused by oligomerization resulting in dimer and higher order aggregates, subvisible, and visible particle formation, and precipitation. The degree of physical degradation can be ascertained using varying techniques depending on the type of degradant of interest. Dimers and higher order soluble aggregates can be quantified using size exclusion chromatography, while subvisible particles may be quantified using light scattering, light obscuration or other suitable techniques.

A protein “retains its chemical stability” in a formulation, if the chemical stability at a given time is such that covalent bonds are not made or broken, resulting in changes to the primary structure of the protein component, e.g., antibody. Changes to the primary structure may result in modifications of the secondary and/or tertiary and/or quaternary structure of the protein and may result in formation of aggregates or reversal of aggregates already formed. Typical chemical modifications can include isomerization, deamidation, N-terminal cyclization, backbone hydrolysis, methionine oxidation, tryptophan oxidation, histidine oxidation, beta-elimination, disulfide formation, disulfide scrambling, disulfide cleavage, and other changes resulting in changes to the primary structure including D-amino acid formation. Chemical instability, i.e., loss of chemical stability, may be interrogated by a variety of techniques including ion-exchange chromatography, capillary isoelectric focusing, analysis of peptide digests and multiple types of mass spectrometric techniques. Chemical stability can be assessed by detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by charge-based methods, such as, but not limited to, ion-exchange chromatography, capillary isoelectric focusing, or peptide mapping.

Loss of physical and/or chemical stability may result in changes to biological activity as either an increase or decrease of a biological activity of interest, depending on the modification and the protein being modified. A protein “retains its biological activity” in a formulation, if the biological activity of the protein at a given time is within about 30% of the biological activity exhibited at the time the formulation was prepared. Activity is considered decreased if the activity is less than 70% of its starting value. Biological assays may include both in vivo and in vitro based assays such as ligand binding, potency, cell proliferation or other surrogate measure of its biopharmaceutical activity.

An antibody used in the practice of the invention, whether a variant or parent antibody, is typically produced by recombinant expression technology. The term “recombinant” indicates that the material (e.g., a nucleic acid or a polypeptide) has been artificially or synthetically (i.e., non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other well known molecular biological procedures. Examples of such molecular biological procedures are found in Maniatis et al., Molecular Cloning. A Laboratory Manual. Cold Spring Harbor Laboratory, Cold Spring Harbor, N.Y. (1982). A “recombinant DNA molecule,” is comprised of segments of DNA joined together by means of such molecular biological techniques.

The term “recombinant protein” or “recombinant polypeptide” as used herein refers to a protein molecule, e.g., an antibody, which is expressed using a recombinant DNA molecule. A “recombinant host cell” is a cell that contains and/or expresses a recombinant nucleic acid.

The term “naturally occurring,” where it occurs in the specification in connection with biological materials such as polypeptides, nucleic acids, host cells, and the like, refers to materials which are found in nature.

The term “control sequence” or “control signal” refers to a polynucleotide sequence that can, in a particular host cell, affect the expression and processing of coding sequences to which it is ligated. The nature of such control sequences may depend upon the host organism. In particular embodiments, control sequences for prokaryotes may include a promoter, a ribosomal binding site, and a transcription termination sequence. Control sequences for eukaryotes may include promoters comprising one or a plurality of recognition sites for transcription factors, transcription enhancer sequences or elements, polyadenylation sites, and transcription termination sequences. Control sequences can include leader sequences and/or fusion partner sequences. Promoters and enhancers consist of short arrays of DNA that interact specifically with cellular proteins involved in transcription (Maniatis, et al., Science 236:1237 (1987)). Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss, et al., Trends Biochem. Sci., 11:287 (1986) and Maniatis, et al., Science 236:1237 (1987)).

A “promoter” is a region of DNA including a site at which RNA polymerase binds to initiate transcription of messenger RNA by one or more downstream structural genes. Promoters are located near the transcription start sites of genes, on the same strand and upstream on the DNA (towards the 5′ region of the sense strand). Promoters are typically about 100-1000 bp in length.

An “enhancer” is a short (50-1500 bp) region of DNA that can be bound with one or more activator proteins (transcription factors) to activate transcription of a gene.

The terms “in operable combination”, “in operable order” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced. For example, a control sequence in a vector that is “operably linked” to a protein coding sequence is ligated thereto so that expression of the protein coding sequence is achieved under conditions compatible with the transcriptional activity of the control sequences.

“Polypeptide” and “protein” are used interchangeably herein and include a molecular chain of two or more amino acids linked covalently through peptide bonds. The terms do not refer to a specific length of the product. Thus, “peptides,” and “oligopeptides,” are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide. The terms also include molecules in which one or more amino acid analogs or non-canonical or unnatural amino acids are included as can be expressed recombinantly using known protein engineering techniques. In addition, proteins can be derivatized as described herein and by other well-known organic chemistry techniques.

A “variant” of a polypeptide (e.g., an immunoglobulin, or an antibody) comprises an amino acid sequence wherein one or more amino acid residues are inserted into, deleted from and/or substituted into the amino acid sequence relative to another polypeptide sequence. Variants can include fusion proteins.

The term “fusion protein” indicates that the protein includes polypeptide components derived from more than one parental protein or polypeptide. Typically, a fusion protein is expressed from a “fusion gene” in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein. Fusion proteins incorporating an antibody or an antigen-binding portion thereof are known.

A “secreted” protein refers to those proteins capable of being directed to the endoplasmic reticulum (ER), secretory vesicles, or the extracellular space as a result of a secretory signal peptide sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage. In some other embodiments, the antibody protein of interest can be synthesized by the host cell as a secreted protein, which can then be further purified from the extracellular space and/or medium.

As used herein “soluble” when in reference to a protein produced by recombinant DNA technology in a host cell is a protein that exists in aqueous solution; if the protein contains a twin-arginine signal amino acid sequence the soluble protein is exported to the periplasmic space in gram negative bacterial hosts, or is secreted into the culture medium by eukaryotic host cells capable of secretion, or by bacterial host possessing the appropriate genes (e.g., the kil gene). Thus, a soluble protein is a protein which is not found in an inclusion body inside the host cell. Alternatively, depending on the context, a soluble protein is a protein which is not found integrated in cellular membranes, or, in vitro, is dissolved, or is capable of being dissolved in an aqueous buffer under physiological conditions without forming significant amounts of insoluble aggregates (i.e., forms aggregates less than 10%, and typically less than about 5%, of total protein) when it is suspended without other proteins in an aqueous buffer of interest under physiological conditions, such buffer not containing an ionic detergent or chaotropic agent, such as sodium dodecyl sulfate (SDS), urea, guanidinium hydrochloride, or lithium perchlorate. In contrast, an insoluble protein is one which exists in denatured form inside cytoplasmic granules (called an inclusion body) in the host cell, or again depending on the context, an insoluble protein is one which is present in cell membranes, including but not limited to, cytoplasmic membranes, mitochondrial membranes, chloroplast membranes, endoplasmic reticulum membranes, etc., or in an in vitro aqueous buffer under physiological conditions forms significant amounts of insoluble aggregates (i.e., forms aggregates equal to or more than about 10% of total protein) when it is suspended without other proteins (at physiologically compatible temperature) in an aqueous buffer of interest under physiological conditions, such buffer not containing an ionic detergent or chaotropic agent, such as sodium dodecyl sulfate (SDS), urea, guanidinium hydrochloride, or lithium perchlorate.

The term “polynucleotide” or “nucleic acid” includes both single-stranded and double-stranded nucleotide polymers containing two or more nucleotide residues. The nucleotide residues comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phosphoraniladate and phosphoroamidate.

The term “oligonucleotide” means a polynucleotide comprising 200 or fewer nucleotide residues. In some embodiments, oligonucleotides are 10 to 60 bases in length. In other embodiments, oligonucleotides are 12, 13, 14, 15, 16, 17, 18, 19, or 20 to 40 nucleotides in length. Oligonucleotides may be single stranded or double stranded, e.g., for use in the construction of a mutant gene. Oligonucleotides may be sense or antisense oligonucleotides. An oligonucleotide can include a label, including a radiolabel, a fluorescent label, a hapten or an antigenic label, for detection assays. Oligonucleotides may be used, for example, as PCR primers, cloning primers or hybridization probes.

A “polynucleotide sequence” or “nucleotide sequence” or “nucleic acid sequence,” as used interchangeably herein, is the primary sequence of nucleotide residues in a polynucleotide, including of an oligonucleotide, a DNA, and RNA, a nucleic acid, or a character string representing the primary sequence of nucleotide residues, depending on context. From any specified polynucleotide sequence, either the given nucleic acid or the complementary polynucleotide sequence can be determined. Included are DNA or RNA of genomic or synthetic origin which may be single- or double-stranded, and represent the sense or antisense strand. Unless specified otherwise, the left-hand end of any single-stranded polynucleotide sequence discussed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences;” sequence regions on the DNA strand having the same sequence as the RNA transcript that are 3′ to the 3′ end of the RNA transcript are referred to as “downstream sequences.”

As used herein, an “isolated nucleic acid molecule” or “isolated nucleic acid sequence” is a nucleic acid molecule that is either (1) identified and separated from at least one contaminant nucleic acid molecule with which it is ordinarily associated in the natural source of the nucleic acid or (2) cloned, amplified, tagged, or otherwise distinguished from background nucleic acids such that the sequence of the nucleic acid of interest can be determined. An isolated nucleic acid molecule is other than in the form or setting in which it is found in nature. However, an isolated nucleic acid molecule includes a nucleic acid molecule contained in cells that ordinarily express the immunoglobulin (e.g., antibody) where, for example, the nucleic acid molecule is in a chromosomal location different from that of natural cells.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of ribonucleotides along the mRNA chain, and also determines the order of amino acids along the polypeptide (protein) chain. The DNA sequence thus codes for the RNA sequence and for the amino acid sequence.

The term “gene” is used broadly to refer to any nucleic acid associated with a biological function. Genes typically include coding sequences and/or the regulatory sequences required for expression of such coding sequences. The term “gene” applies to a specific genomic or recombinant sequence, as well as to a cDNA or mRNA encoded by that sequence. Genes also include non-expressed nucleic acid segments that, for example, form recognition sequences for other proteins. Non-expressed regulatory sequences including transcriptional control elements to which regulatory proteins, such as transcription factors, bind, resulting in transcription of adjacent or nearby sequences.

“Expression of a gene” or “expression of a nucleic acid” means transcription of DNA into RNA (optionally including modification of the RNA, e.g., splicing), translation of RNA into a polypeptide (possibly including subsequent post-translational modification of the polypeptide), or both transcription and translation, as indicated by the context.

An expression cassette is a typical feature of recombinant expression technology. The expression cassette includes a gene encoding a protein of interest, e.g., a gene encoding an antibody sequence, such as an immunoglobulin light chain and/or heavy chain sequence. A eukaryotic “expression cassette” refers to the part of an expression vector that enables production of protein in a eukaryotic cell, such as a mammalian cell. It includes a promoter, operable in a eukaryotic cell, for mRNA transcription, one or more gene(s) encoding protein(s) of interest and a mRNA termination and processing signal. An expression cassette can usefully include among the coding sequences, a gene useful as a selective marker. In the expression cassette promoter is operably linked 5′ to an open reading frame encoding an exogenous protein of interest; and a polyadenylation site is operably linked 3′ to the open reading frame. Other suitable control sequences can also be included as long as the expression cassette remains operable. The open reading frame can optionally include a coding sequence for more than one protein of interest.

As used herein the term “coding region” or “coding sequence” when used in reference to a structural gene refers to the nucleotide sequences which encode the amino acids found in the nascent polypeptide as a result of translation of an mRNA molecule. The coding region is bounded, in eukaryotes, on the 5′ side by the nucleotide triplet “ATG” which encodes the initiator methionine and on the 3′ side by one of the three triplets which specify stop codons (i.e., TAA, TAG, TGA).

Recombinant expression technology typically involves the use of a recombinant expression vector comprising an expression cassette and a mammalian host cell comprising the recombinant expression vector with the expression cassette or at least the expression cassette, which may for example, be integrated into the host cell genome.

The term “vector” means any molecule or entity (e.g., nucleic acid, plasmid, bacteriophage or virus) used to transfer protein coding information into a host cell.

The term “expression vector” or “expression construct” as used herein refers to a recombinant DNA molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired. Such techniques are well known in the art. (See, e.g., Goodey, Andrew R.; et al., Peptide and DNA sequences, U.S. Pat. No. 5,302,697; Weiner et al., Compositions and methods for protein secretion, U.S. Pat. Nos. 6,022,952 and 6,335,178; Uemura et al., Protein expression vector and utilization thereof, U.S. Pat. No. 7,029,909; Ruben et al., 27 human secreted proteins, US 2003/0104400 A1). For expression of multi-subunit proteins of interest, separate expression vectors in suitable numbers and proportions, each containing a coding sequence for each of the different subunit monomers, can be used to transform a host cell. In other embodiments, a single expression vector can be used to express the different subunits of the protein of interest.

The term “host cell” means a cell that has been transformed, or is capable of being transformed, with a nucleic acid and thereby expresses a gene or coding sequence of interest. The term includes the progeny of the parent cell, whether or not the progeny is identical in morphology or in genetic make-up to the original parent cell, so long as the gene of interest is present. Any of a large number of available and well-known host cells may be used in the practice of this invention to obtain antibody variants, although mammalian host cells capable of post-translationally glycosylating antibodies are preferred. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Modifications can be made at the DNA level, as well. The peptide-encoding DNA sequence may be changed to codons more compatible with the chosen host cell. Codons can be substituted to eliminate restriction sites or to include silent restriction sites, which may aid in processing of the DNA in the selected host cell. Next, the transformed host is cultured and purified. Host cells may be cultured under conventional fermentation conditions so that the desired compounds are expressed. Such fermentation conditions are well known in the art.

Within these general guidelines, microbial host cells in culture, such as bacteria (such as Escherichia coli sp.), and yeast cell lines (e.g., Saccharomyces, Pichia, Schizosaccharomyces, Kluyveromyces) and other fungal cells, algal or algal-like cells, insect cells, plant cells, that have been modified to incorporate humanized glycosylation pathways, can also be used to produce fully functional glycosylated antibody. However, mammalian (including human) host cells, e.g., CHO cells and HEK-293 cells, are particularly useful.

Examples of useful mammalian host cell lines are Chinese hamster ovary cells, including CHO-K1 cells (e.g., ATCC CCL61), CHO-S, DXB-11, DG-44, and Chinese hamster ovary cells/-DHFR (CHO, Urlaub et al, Proc. Natl. Acad. Sci. USA 77: 4216 (1980)); monkey kidney CV1 line transformed by SV40 (COS-7, ATCC CRL 1651); human embryonic kidney line (293 or 293 cells subcloned for growth in suspension culture (Graham et al, J. Gen Virol. 36: 59 (1977)); baby hamster kidney cells (BHK, ATCC CCL 10); mouse Sertoli cells (TM4, Mather, Biol. Reprod. 23: 243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TRI cells (Mather et al., Annals N.Y Acad. Sci. 383: 44-68 (1982)); MRC 5 cells or FS4 cells; or mammalian myeloma cells, e.g., NSO or sp2/0 mouse myeloma cells.

“Cell,” “cell line,” and “cell culture” are often used interchangeably and all such designations herein include cellular progeny. For example, a cell “derived” from a CHO cell is a cellular progeny of a Chinese Hamster Ovary cell, which may be removed from the original primary cell parent by any number of generations, and which can also include a transformant progeny cell. Transformants and transformed cells include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

Host cells are transformed or transfected with the above-described nucleic acids or vectors for production of polypeptides (including antigen binding proteins, such as antibodies) and are cultured in conventional nutrient media modified as appropriate for inducing promoters, selecting transformants, or amplifying the genes encoding the desired sequences. In addition, novel vectors and transfected cell lines with multiple copies of transcription units separated by a selective marker are particularly useful for the expression of polypeptides, such as antibodies.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA may recombine with that of the cell by physically integrating into a chromosome of the cell, or may be maintained transiently as an episomal element without being replicated, or may replicate independently as a plasmid. A cell is considered to have been “stably transformed” when the transforming DNA is replicated with the division of the cell.

The inventive high throughput method for selecting an antibody variant amino acid sequence of interest from a plurality of antibody variant amino acid sequences involves the transfected mammalian cells under physiological conditions allowing the cells to express and secrete recombinant antibodies.

The host cells can be usefully grown in batch culture, fed-batch culture, intensified fed-batch culture (product retention perfusion), or in continuous culture systems employing liquid aqueous medium. Mammalian cells, such as CHO and BHK cells, are generally cultured as suspension cultures. That is to say, the cells are suspended in a liquid cell culture medium, rather than adhering to a solid support. In other embodiments, the mammalian host cells can be cultured on solid or semi-solid aqueous culture medium, for example, containing agar or agarose, to form a medium, carrier (or microcarrier) or substrate surface to which the cells adhere and form an adhesion layer. Another useful mode of production is a hollow fiber bioreactor with an adherent cell line. Porous microcarriers can be suitable and are available commercially, sold under brands, such as Cytoline®, Cytopore® or Cytodex® (GE Healthcare Biosciences).

“Cell culture medium” or “culture medium,” used interchangeably, is defined, for purposes of the invention, as a sterile medium suitable for growth of cells, and preferably animal cells, more preferably mammalian cells (e.g., CHO cells), in in vitro cell culture. Any medium capable of supporting growth of the appropriate cells in cell culture can be used. Suitably, the culture medium has an osmolality of between 210 and 650 mOsm, preferably 270 to 450 mOsm, more preferably 310 to 350 mOsm and most preferably 320 mOsm. Preferably, the osmolality of the cell culture supernatant is maintained within one or more of these ranges throughout the culturing of host cells. The cell culture medium can be based on any basal medium such as DMEM, or RPMI generally known to the skilled worker. Commercially available media such as ExpiCHO™ Expression Medium (ThermoFisher Scientific), Ham's F10 (Sigma), Ham's F12, Medium 199, McCoy, Minimal Essential Medium ((MEM), (Sigma-Aldrich), RPMI-1640 (Sigma), and Dulbecco's Modified Eagle's Medium ((DMEM), Sigma-Aldrich) are suitable for culturing various host cells. In addition, any of the media described in Ham et al., Meth. Enz. 58: 44 (1979), Barnes et al., Anal. Biochem. 102: 255 (1980), U.S. Pat. Nos. 4,767,704; 4,657,866; 4,927,762; 4,560,655; or 5,122,469; WO90103430; WO 87/00195; or U.S. Pat. Re. No. 30,985 may be used as culture media for the host cells, or modified appropriately to suit the cell line employed. Other examples include HyClone ActiPro™ and Lonza PowerCHO-2™. The basal medium can comprise a number of ingredients, including amino acids, vitamins, organic and inorganic salts, and sources of carbohydrate, each ingredient being present in an amount which supports the cultivation of a cell which is generally known to the person skilled in the art. The medium can contain auxiliary substances, such as buffer substances like sodium bicarbonate, antioxidants, stabilizers to counteract mechanical stress, or protease inhibitors. Any of these media may be supplemented as necessary with hormones and/or other growth factors (preferably recombinantly produced), such as insulin, insulin-like growth factor (IGF)-1, transferrin, or epidermal growth factor; salts, such as sodium chloride, calcium, magnesium, and phosphate; buffers, such as HEPES and/or sodium bicarbonate; nucleotides, such as adenosine and thymidine; antibiotics, such as gentamicin, neomycin, tetracycline, puromycin, or kanamycin; trace elements (defined as inorganic compounds usually present at final concentrations in the micromolar range); and glucose or an equivalent carbon and/or energy source, such that the physiological conditions of the cell in, or on, the medium promote expression of the protein of interest by the host cell; any other necessary supplements may also be included at appropriate concentrations that would be known to those skilled in the art.

Historically, mammalian cells have been cultured in media containing mammalian serum. The culture medium can include a suitable amount of serum such a fetal bovine serum (FBS). The term “serum-comprising” as applied to cell culture medium includes any cell culture medium that does contain serum. However, such media are incompletely defined and carry the risk of infection, therefore, preferably, the host cells can be adapted for culture in serum-free medium. The term “serum-free” as applied to medium includes any cell culture medium that does not contain serum. By “serum-free”, it is understood that the medium has preferably less than 0.1% (v/v) serum and more preferably less than 0.01% (v/v) serum. The term “serum” refers to the fluid portion of the blood obtained after removal of the fibrin clot and blood cells.

Those in the art have devised “protein-free” media that are either completely free of any protein or at least are free of any protein that is not recombinantly produced. Due to the labile nature of Factor VIII, the productivity of the engineered host cells is severely reduced under protein-free conditions. Human serum albumin is commonly used as a serum-free culture supplement for the production of recombinant proteins. The albumin itself stabilizes the FVIII and the impurities present in serum-derived albumin preparations may also contribute to the stabilizing effect of albumin. Factors such as lipoprotein have been identified as a replacement for human serum albumin for the production of recombinant Factor VIII under serum-free conditions.

Useful cell culture media include those disclosed in U.S. Pat. No. 6,171,825 (Chan et al., Preparation of recombinant factor VIII in a protein free medium, Bayer, Inc.) and U.S. Pat. No. 6,936,441 (Reiter et al., Recombinant cell clones having increased stability and methods of making and using the same, Baxter A G). The medium of U.S. Pat. No. 6,171,825 (Chan et al.) comprises modified Dulbecco's Minimum Essential Medium and Ham's F-12 Medium (50:50, by weight) supplemented with recombinant insulin, iron, a polyol, copper and optionally other trace metals.

If insulin is used, it should be recombinant and can be obtained commercially as “Nucellin” insulin (Eli Lilly. It can be added at 0.1 to 20 μg/mL (preferably 5-15 μg/mL, or about 10 μg/mL). The iron is preferably in the form of Fe²⁺ ions, for example provided as FeSO₄EDTA, and can be present at 5-100 μM (preferably about 50 μM). Suitable polyols include non-ionic block copolymers of poly(oxyethylene) and poly(oxypropylene) having molecular weights ranging from about 1000 to about 16,000. A particularly preferred polyol is Pluronic F-68 (BASF Wyandotte), which has an average molecular weight of 8400 and consists of a center block of poly(oxypropylene) (20% by weight) and blocks of poly(oxyethylene) at both ends. It is also available as Synperonic F-68 from Unichema Chemie BV. Others include Pluronics F-61, F-71 and F-108. Copper (Cu.sup.2+) may be added in an amount equivalent to 50-800 nM CuSO4, preferably 100-400 nM, conveniently about 250 nM. The inclusion of a panel of trace metals such as manganese, molybdenum, silicon, lithium and chromium can lead to further increases in Factor VIII production. BHK cells grow well in this protein-free basal medium.

The medium of U.S. Pat. No. 6,936,441 (Reiter et al.) is particularly well suited to the culturing of CHO cells but may be used with other cells as well. The medium of U.S. Pat. No. 6,936,441 is also based on a 50/50 mixture of DMEM and Ham's F12 but includes soybean peptone hydrolysate or yeast extract at between 0.1 and 100 g/L, preferably between 1 and 5 g/L. As a particularly preferred embodiment, soybean extract, e.g. soybean peptone, may be used. The molecular weight of the soybean peptone can be less than 50 kDa, preferably less than 10 kDa. The addition of ultrafiltered soybean peptone having an average molecular weight of 350 Dalton has proven particularly advantageous for the productivity of the recombinant cell lines. It is a soybean isolate having a total nitrogen content of about 9.5% and a free amino acid content of about 13%, or about 7-10%.

Another useful embodiment of a cell culture medium has the following composition: synthetic minimum medium (e.g. 50/50 DMEM/Ham's F12) 1 to 25 g/L; soybean peptone 0.5 to 50 g/L; L-glutamine 0.05 to 1 g/L; NaHCO₃ 0.1 to 10 g/L; ascorbic acid 0.0005 to 0.05 g/L; ethanolamine 0.0005 to 0.05; and sodium selenite 1 to 15 μg/L. Optionally, a “defoaming” or “anti-foaming” agent can be added to the culture medium. Examples include, a silicone antifoam agent, or a non-ionic surface-active agent such as a polypropylene glycol (e.g. Pluronic F-61, Pluronic F-68, Pluronic F-71 or Pluronic F-108). Another example of a useful commercially available anti-foaming agent is Ex-Cell® Antifoam (Sigma-Aldrich, Inc., St. Louis, Mo.; Product No. 59920C). The anti-foam agent is generally applied to protect the cells from the negative effects of aeration (“sparging”), since without the addition of a surface-active agent the rising and bursting air bubbles may damage those cells that are at the surface of the air bubbles.

The amount of non-ionic surface-active agent can range between 0.05 and 10 g/L, preferably between 0.1 and 5 g/L. Furthermore, the medium can also contain cyclodextrin or a derivative thereof. The serum- and protein-free medium can also contain a protease inhibitor, such as a serine protease inhibitor, which is suitable for tissue culture and which is of synthetic or vegetable origin. Non-ionic surfactants or antifoaming agents, if present in the cell culture medium, are preferably removed from the buffer in which the antibodies are dissolved before any affinity chromatography steps, lest they interfere.

In another embodiment of a cell culture medium, the following amino acid mixture is can be added to the above-mentioned medium: L-asparagine (0.001 to 1 g/L; preferably 0.01 to 0.05 g/L; particularly preferably 0.015 to 0.03 g/l), L-cysteine (0.001 to 1 g/L; preferably 0.005 to 0.05 g/L; particularly preferably 0.01 to 0.03 WI), L-cysteine (0.001 to 1 g/L; preferably 0.01 to 0.05 g/L; particularly preferably 0.015 to 0.03 g/L), L-proline (0.001 to 1.5 g/L; preferably 0.01 to 0.07 g/L; particularly preferably 0.02 to 0.05 g/L), L-tryptophan (0.001 to 1 g/L; preferably 0.01 to 0.05 g/L; particularly preferably 0.015 to 0.03 g/L) and L-glutamine (0.05 to 10 g/L; preferably 0.1 to 1 g/L). These amino acids may be added to the medium individually or in combination. The combined addition of the amino acid mixture containing all of the above-mentioned amino acids is particularly preferred.

In one embodiment, a serum- and protein-free medium is used additionally containing a combination of the above-mentioned amino acid mixtures and purified, ultrafiltered soybean peptone hydrolysate.

Nutrient supplements such as yeast hydrolysate or various plant-based hydrolysates can be included in the medium, if desired. In some embodiments, the aqueous medium is liquid, such that the host cells are cultured in a cell suspension within the liquid medium. Alternate media capable of supporting CHO cell growth and productivity of antibody can be used interchangeably with the media used in the working example described herein. The possibilities are numerous and could include commercial media made by Sigma Aldrich, Sartorius or Irvine Scientific, as well as, media especially formulated for a variety of suitable host cell types.

The term “hydrolysate” includes any digest of an animal derived or plant derived source material, or extracts derived from yeast, bacteria, or plants, e.g., “soy hydrolysate,” which can be a highly purified soy hydrolysate, a purified soy hydrolysate or crude soy hydrolysate.

A further suitable cell culture medium is the oligopeptide-free medium disclosed in US 2007/0212770 A1 (Grillberger et al., Oligopeptide free cell culture media; Baxter International Inc., Baxter Healthcare S.A.), but any suitable cell culture medium that provides physiological conditions permitting the expression of antibody proteins by the host cells can be employed, including other media described in the Examples herein.

The term “inoculation of the cells into the cell culture medium” refers to the step of contacting the cells with the cell culture medium under conditions which are suitable for growth and proliferation of the cells.

The cell culture contemplated herein may be any cell culture independently of the kind and nature of the cultured cells and the growth phase of the cultured cells, e.g. adherent or non-adherent cells; growing, or growth-arrested cells.

The term “sterile,” as used herein, refers to a substance that is free, or essentially free, of microbial and/or viral contamination. In this respect the “contaminant” means a material that is different from the desired components in a preparation being a cell culture medium or at least a component of a cell culture medium. In the context of “sterile filtration”, the term sterile filtration is a functional description that a preparation is filtered through a sterile filter (with a pore size of 0.2 μm or less) to remove bacterial and/or mycoplasma contaminants.

“Batch filtration,” otherwise known as “batch wise filtration” or filtration done in batch mode, refers herein to a process wherein a specific total amount or volume of a preparation, being a cell culture medium or at least a component of a cell culture medium, is filtered through a virus filter in one batch dependent on the capacity of the virus filter and wherein the filtration process is finalized before the filtrate is directed or fed to the process in which it is used or consumed.

The term “continuous filtration” or “online filtration” or “in line filtration” refers to a filtration process, wherein the specific total amount or volume of a preparation, being a cell culture medium or at least a component of a cell culture medium, is filtered through the virus filter continuously dependent on the capacity of the virus filter and wherein the filtration process is still going on when the filtrate is already directed or fed to the process in which it is used or consumed.

The “cell culture supernatant” is the extracellular medium in which the mammalian cells are cultured. This medium is not to be confused with feed medium that may be added to the culture after inoculation of the cells into the cell culture medium and cell growth has been commenced. A “cell culture” means the cell culture supernatant and the mammalian cells cultured therein. Conventionally, mammalian cells are cultured at 37° C.±1° C.

By “culturing at” or “maintaining at” a temperature, is meant that the temperature to which the process control systems are set, in other words the intended, target temperature, pH, oxygenation level. The culture conditions, such as temperature (typically, but not necessarily, about 37° C.), pH (typically, but not necessarily, a cell culture medium is maintained within the range of about pH 6.5-7.5, as modified consistent with the present invention), oxygenation, and the like, will be apparent to the ordinarily skilled artisan. Clearly, there will be small variations of the temperature of a culture over time, and from location to location through the culture vessel. Digital control units and sensory monitors are available commercially or can be constructed by the skilled artisan. Alternative digital control units (DCU) control and monitor the cell culture process are available commercially, made by companies such as B. Braun, New Brunswick, or Sartorius. For in-flask batch culture with shaker, numerous models of suitable cell culture incubators with built-in environmental controls (e.g., CO₂ and Multigas CO₂/O₂ controls) are commercially available, e.g., by Thermo Fisher Scientific.

“Culturing at” or “maintaining at” a temperature that is set at X±Y° C., means that the set point is at a value of from X+Y° C. to X−Y° C. For example, where X is 37.0±0.9° C., the set-point is set at a value of from 37.9 to 36.1° C. For each of the preferred values of X, e.g., X=31, X=32, X=33, X=34, X=35, X=36, or X=37, the set-point is at a value within the range X±0.9° C., ±0.8° C., ±0.7° C., ±0.6° C., ±0.5° C., ±0.4° C., ±0.3° C., ±0.2° C., or ±0.1° C. (See, e.g., Oguchi et al., pH Condition in temperature shift cultivation enhances cell longevity and specific hMab productivity in CHO culture, Cytotechnology. 52(3):199-207 (2006); Al-Fageeh et al., The cold-shock response in cultured mammalian cells: Harnessing the response for the improvement of recombinant protein production, Biotechnol. Bioeng. 93:829-835 (2006); Marchant, R. J. et al., Metabolic rates, growth phase, and mRNA levels influence cell-specific antibody production levels from in vitro cultured mammalian cells at sub-physiological temperatures, Mol. Biotechnol. 39:69-77 (2008)).

For any given set-point, slight variations in temperature may occur. Typically, such variation may occur because heating and cooling elements are only activated after the temperature has deviated somewhat from the set-point. In that case, the set-point is X±Y and the heating or cooling element is activated when the temperature varies by ±Z° C., as appropriate. Typically, the permissible degree of deviation of the temperature from the set-point before heating or cooling elements are activated may be programmed in the process control system. Temperature may be controlled to the nearest ±0.5° C., ±0.4° C., ±0.3° C., ±0.2° C., or even ±0.1° C. by heating and cooling elements controlled by thermostats. Larger differentials in temperature may also be programmed, such as ±1.0° C., ±0.9° C., ±0.8° C., ±0.7° C., or ±0.6° C. The temperature may also be controlled by immersion of the culture vessel in a heating bath at a particular temperature. Conceivably, there is no variation from the set-point because the heating is applied continually. Another source of variation arises due to measurement error in the temperature of the cell culture supernatant. Typical thermometers used in cell culture equipment may have a variability of ±0.3° C., or ±0.2° C., or even ±0.1° C.

Where the temperature set-point is set at a value within the range X±Y° C., and the tolerance of the temperature is ±Z° C. (i.e. a heater or cooler is activated when the temperature deviates by ±Z° C., as appropriate) this can also be expressed as a set-point of (X−Y to X+Y)±Z° C. For each possible value of X, all combinations of ±Y° C. and ±Z° C., as indicated above, are envisaged.

“Culturing at” or “maintaining at” a set point of a particular desired pH value, means that the process control systems are set to that desired pH value, in other words that the set point of pH is the intended, target, pH. “Culturing at” or “maintaining at” a pH that is set at X±Y, means that the set point is at a value of from X+Y to X−Y pH units. For each of the preferred values of X, the set-point is at a value within the range pH X±0.05, ±0.04, ±0.03, ±0.02 or ±0.01.

Where the pH set-point is set at a value within the range X±Y, and the tolerance is ±Z, this can also be expressed as a set-point of (X−Y to X+Y)±Z. For each possible value of X, all combinations of ±Y and ±Z, as indicated above.

For any given pH set-point, slight variations in pH may occur. Typically, such variation can occur because means which control pH are only activated after the pH has deviated somewhat from the set-point. Typically, the pH is controlled to the nearest ±0.05, ±0.04, ±0.03, ±0.02, or ±0.01. Typically, sparging with CO₂ provides additional acid in mammalian cell culture. Liquid acids, e.g., HCl or H₃PO₄, are commonly used in microbial cultures. Sodium carbonate is usually the source of added alkali used to maintain pH for mammalian cell culture, and NH₄OH is often selected to add alkali in microbial culture.

The cell culture supernatant typically has a CO₂ concentration of 1 to 10% (v/v), for example 4.0-9.0% (v/v), 5.5-8.5% (v/v) or about 6-8% (v/v). Conventionally, CO₂ concentration is higher than this due to the CO₂ produced by the cells not being removed from the cell culture supernatant. Maintaining the CO₂ concentration at 10% or lower is reported to increase the yield of recombinant protein; it helps the dCO2 (or pCO₂) to be kept low if the feed medium is degassed (for example by bubbling air through it) as well as the cell culture supernatant in the bioreactor being sparged. (See, Giovagnoli et al., Cell Culture Processes, US2009/0176269, US2016/0244506, U.S. Pat. No. 9,359,629, EP2235197, EP2574676).

Ways of monitoring culture parameters of temperature, pH and CO₂ concentration are well known in this art and generally rely on probes that are inserted into the bioreactor, or included in loops through which the culture medium is circulated, or inserted into extracted samples of culture medium. Suitable monitoring equipment and appropriate alternatives are commercially available or can be constructed by the skilled artisan. Alternative gas analyzers are commercially available, such as RapidLab® 248 (Siemens) and others made by Nova® Biomedical, Radiometer America and Roche Diagnostics. Mass flow controllers can also be used to control gas and liquid additions in labs that are properly equipped. A suitable in-line dCO₂ (or pCO₂) sensor and its use are described in Pattison et al (2000) Biotechnol. Frog. 16:769-774. A suitable in-line pH sensor is Mettler Toledo InPro 3100/125/Pt100 (Mettler-Toledo Ingold, Inc., Bedford, Mass.). A suitable off-line system for measuring dCO₂ (or pCO₂), in addition to pH and pO₂ is the BioProfile pHOx (Nova Biomedical Corporation, Waltham Mass.). In this system, or dCO2 (or pCO₂) is measured by potentiometric electrodes within the range 3-200 mmHg with an imprecision resolution of 5%. The pH may be measured in this system at a temperature of 37° C., which is close to the temperature of the cell culture supernatant in the bioreactor. Ways of altering the specified parameter in order to keep it at the predefined level are also well known. For example, keeping the temperature constant usually involves heating or cooling the bioreactor or the feed medium (if it is a fed-batch or continuous process); keeping the pH constant usually involves choosing and supplying enough of an appropriate buffer (typically bicarbonate) and adding acid, such as hydrochloric acid, or alkali, such as sodium hydroxide, sodium carbonate or a mixture thereof, to the feed medium as necessary; and keeping the CO₂ concentration constant usually involves adjusting the sparging rate (see further below), or regulating the flow of CO₂ in the head space. It is possible that the calibration of an in-line pH probe may drift over time, such as over periods of days or weeks, during which the cells are cultured. In that event, it may be beneficial to reset the in-line probe by using measurements obtained from a recently calibrated off-line probe. A suitable off-line probe is the BioProfile pHOx (Nova Biomedical Corporation, Waltham Mass.).

Mammalian cell cultures need oxygen for the cells to grow. Normally, this is provided by forcing oxygen into the culture through injection ports. It is also necessary to remove the CO₂ that accumulates due to the respiration of the cells. This is achieved by “sparging,” i.e., passing a gas through the bioreactor in order to entrain and flush out the CO₂. Conventionally, this can also be done using oxygen. However, the inventors have found that it is advantageous to use air instead. It has been found that usually a conventional inert gas such as nitrogen is less effective at sparging CO₂ than using air. Given that air is about 20% (v/v) oxygen, one might have thought that five times as much air would be used. However, this has been found to be inadequate in large scale cultures, particularly in cultures at 2500 L scale. In a 2500 L bioreactor, 7 to 10 times as much air, preferably about 9 times as much air, is used. For example, under standard conditions, the 2500 L bioreactor is sparged with O₂ at a 10-μm bubble size at a rate of 0.02 VVH (volume O₂ per volume of culture per hour). The same 2500 L bioreactor used according to the method of the invention would be sparged with air at a 10-μm bubble size at a rate of 0.18 VVH.

Hence, the use of surprisingly high volumes of air has been found to provide adequate oxygen supply and to remove the unwanted CO₂. Flushing the bioreactor head space with air is also a useful mechanism for removing excess CO₂.

During production phase, it is preferred to remove CO₂ by air sparging, as described above. This is especially the case when using bioreactors of large capacity, in which the cell culture supernatant would otherwise accumulate CO₂ to deleteriously high levels. However, at the beginning of culture, or in small scale culture, such as at 1-L or 2.5-L scale, the head space may be overlayed with CO₂. Under such conditions, low levels of dCO₂ (or pCO₂) can still be achieved. Overlaying the headspace with CO₂ may also be used to reduce the pH to the set-point, if the pH is too basic.
In accordance with inventive method, the culturing of a plurality of mammalian host cells can be any conventional type of culture, such as batch, fed-batch, intensified fed-batch, or continuous. Suitable continuous cultures included repeated batch, chemostat, turbidostat or perfusion culture. For purposes of the present invention, the desired scale of the recombinant expression will be dependent on the type of expression system and the quantity of different theoretical antibody variants to be studied. As noted herein, typically, 100 milligrams of total antibody protein will suffice, requiring only a batch cell culture of 20 mL to 500 mL; while larger scale culture batches or continuous cell culture methods can be employed, larger volumes are typically not cost-effective.

A batch culture starts with all the nutrients and cells that are needed, and the culture proceeds to completion, i.e. until the nutrients are exhausted or the culture is stopped for some reason.

A fed-batch culture is a batch process in the sense that it starts with the cells and nutrients but it is then fed with further nutrients in a controlled way. The fed-batch strategy is typically used in bio-industrial processes to reach a high cell density in the bioreactor. The feed solution is usually highly concentrated to avoid dilution of the bioreactor. The controlled addition of the nutrient directly affects the growth rate of the culture and allows one to avoid overflow metabolism (formation of metabolic by-products) and oxygen limitation (anaerobiosis). In most cases the growth-limiting nutrient is glucose which is fed to the culture as a highly concentrated glucose syrup (for example 500-850 g/L).

Different strategies can be used to control the growth in a fed-batch process. For example, any of dissolved oxygen tension (DOT, pO2), oxygen uptake rate (OUR), glucose concentration, lactate concentration, pH and ammonia concentration can be used to monitor and control the culture growth by keeping that parameter constant. In a continuous culture, nutrients are added and, typically, medium is extracted in order to remove unwanted by-products and maintain a steady state. Suitable continuous culture methods are repeated batch culture, chemostat, turbidostat and perfusion culture.

CHO cells, for example, may be cultured in a stirred tank or an airlift tank that is perfused with a suitable medium at a perfusion rate of from 1 to 10 volume exchanges per day and at an oxygen concentration of between 40% and 60%, preferably about 50%. Moreover, the cells may be cultured by means of the chemostat method, using the preferred pH value given above, an oxygen concentration of between 10% and 60% (preferably about 20%) and a dilution rate D of 0.25 to 1.0, preferably about 0.5.

In a repeated batch culture, also known as serial subculture, the cells are placed in a culture medium and grown to a desired cell density. To avoid the onset of a decline phase and cell death, the culture is diluted with complete growth medium before the cells reach their maximum concentration. The amount and frequency of dilution varies widely and depends on the growth characteristics of the cell line and convenience of the culture process. The process can be repeated as many times as required and, unless cells and medium are discarded at subculture, the volume of culture will increase stepwise as each dilution is made. The increasing volume may be handled by having a reactor of sufficient size to allow dilutions within the vessel or by dividing the diluted culture into several vessels. The rationale of this type of culture is to maintain the cells in an exponentially growing state. Serial subculture is characterized in that the volume of culture is always increasing stepwise, there can be multiple harvests, the cells continue to grow and the process can continue for as long as desired.

In the chemostat and turbidostat methods, the extracted medium contains cells. Thus, the cells remaining in the cell culture vessel must grow to maintain a steady state. In the chemostat method, the growth rate is typically controlled by controlling the dilution rate i.e. the rate at which fresh medium is added. The cells are cultured at a sub-maximal growth rate, which is achieved by restricting the dilution rate. The growth rate is typically high. In contrast, in the turbidostat method, the dilution rate is set to permit the maximum growth rate that the cells can achieve at the given operating conditions, such as pH and temperature.

In an intensified fed-batch culture, culture vessels, reactors or chambers, of any of various capacities are used to grow suspensions of mammalian host cells, e.g., CHO cells. Each culture vessel is connected via inlets to an array of porous tangential flow filters which in turn are connected via outlets back to the culture vessel. After cell growth, the suspensions of host cells and growth medium are pumped through the array of porous tangential flow filters to concentrate the cell suspension. The cell suspension is recycled through the filters and culture vessel allowing a portion of the old growth medium (and its serum components, if any) to be removed. A supply of fresh sterile serum-free expression medium is added to the concentrated cell suspension to maintain a nominal volume in the culture vessel. The recombinant protein of interest, e.g., an antibody, is produced subsequently by the host cells suspended in the expression medium and is secreted by the cells into the expression medium from which it can be harvested by standard techniques. (See, e.g., Zijlstra et al., Process for the culturing of cells, U.S. Pat. Nos. 8,119,368, 8,222,001, 8,440,458).

In a perfusion or continuous culture, the extracted medium is depleted of cells, because most of the cells are retained in the culture vessel, for example, by being retained on a membrane through which the extracted medium flows. However, typically such a membrane retains 100% of cells, and so a proportion are removed when the medium is extracted. Alternatively, sonic cell separation technology achieves separation of cells from the media matrix with high-frequency, resonant ultrasonic waves rather than using a physical barrier, unlike tangential-flow filtration (TFF) or alternating tangential flow filtration (ATF); the cells are held back using an acoustic field as the bioprocess fluid flows through an open channel. The use of acoustic waves allows differentiation of particles of equal size, and thus the technology can be used for the separation of particles from the nano- to macro-scales. (See, e.g., Challenger, C. A., An acoustic wave-based technology for cell harvesting applications may help enable continuous manufacturing, BioPharm International 30(9):30 (2017)). Regardless of the technology employed to separate the cells from the extracted medium, it may not be crucial to operate perfusion cultures at very high growth rates, as the majority of the cells are retained in the culture vessel.

Continuous cultures, particularly repeated batch, chemostat and turbidostat cultures, are typically operated at high growth rates. According to common practice, it is typical to seek to maintain growth rates at maximum, or close to maximum, in an effort to obtain maximum volumetric productivity. Volumetric productivity is measured in units of protein quantity or activity per volume of culture per time interval. Higher cell growth equates to a higher volume of culture being produced per day and so is conventionally considered to reflect a higher volumetric productivity. A suitable fully continuous process can have a perfusion bioreactor coupled to recombinant protein harvesting and protein purification steps, for example, a multi-column chromatography capture step, followed by flow-through virus inactivation, multi-column intermediate purification, a flow-through membrane adsorber polishing step, continuous virus filtration and a final ultrafiltration step operated in continuous mode. (See, e.g., Crowley et al., Process for cell culturing by continuous perfusion and alternating tangential flow, U.S. Pat. No. 8,206,981).

The cell density is commonly monitored in cell cultures. In principle, a high cell density would be considered to be desirable since, provided that the productivity per cell is maintained, this should lead to a higher productivity per bioreactor volume. However, increasing the cell density can actually be harmful to the cells, and the productivity per cell is reduced. There is therefore a need to monitor cell density. To date, in mammalian cell culture processes, this has been done by extracting samples of the culture and analyzing them under a microscope or using a cell counting device such as the CASY TT device sold by Scharfe System GmbH, Reutlingen, Germany. It can be advantageous to analyze the cell density by means of a suitable probe introduced into the bioreactor itself (or into a loop through which the medium and suspended cells are passed and then returned to the bioreactor). Such probes are available commercially from Aber Instruments, for example the Biomass Monitor 220, 210 220 or 230. The cells in the culture act as tiny capacitors under the influence of an electric field, since the non-conducting cell membrane allows a build-up of charge. The resulting capacitance can be measured; it is dependent upon the cell type and is directly proportional to the concentration of viable cells. A probe of 10 to 25 mm diameter uses two electrodes to apply a radio frequency field to the biomass and a second pair of electrodes to measure the resulting capacitance of the polarized cells. Electronic processing of the resulting signal produces an output which is an accurate measurement of the concentration of viable cells. The system is insensitive to cells with leaky membranes, the medium, gas bubbles and debris. Alternatively, cell viability can be measured by use of a vital dye (or vital stain) to stain small-aliquot samples of culture sampled periodically, and microscopically enumerated to determine viable cell count. For example Trypan blue is a vital dye commonly used for this purpose. Automated cell counters supplied by Beckman (e.g., Vi-Cell™ XR) and other companies are available. Examples include cell counting instruments made by other manufacturers, e.g., Nova Biomedical, Olympus, Thermo Fisher Scientific and Eppendorf. Cells can also be counted using flow cytometry or manually by using a hemocytometer.

Typically, a viable cell density can be used from 1.0×10⁶ to 2.0×10⁷, or up to about 5×10⁷ cells/mL. It is known that increasing the concentration of cells toward the higher end of the preferred ranges can improve volumetric productivity. Nevertheless, ranges of cell density including any of the above point values as lower or higher ends of a range are envisaged.

The culture is typically carried out in a bioreactor, which is usually a stainless steel, glass or plastic vessel of 0.01 (i.e., 10-mL) to 10000 (ten thousand) litres capacity, for example, 0.01, 0.015, 0.10, 0.25, 0.30, 0.35, 1, 2, 5, 10, 15, 20, 25, 30, 50, 75, 100, 500, 1000, 2500, 5000 or 8000 liters. The vessel is usually rigid but flexible plastic bags or bioreactor liners can be used. These flexible plastic bioreactor bags and liners are generally of the “single use” type.

Upon culturing the host cells, the recombinant polypeptide or protein, can be produced intracellularly, in the periplasmic space, or, preferably, directly secreted into the medium. Harvesting the recombinant protein involves separating it from particulate matter that can include host cells, cell aggregates, and/or lysed cell fragments, into a cell-free supernatant fraction that is free of host cells and cellular debris. Such cellular debris is removed, for example, by centrifugation or microfiltration. After the recombinant protein, e.g., recombinant antibodies, is separated from the host cells and/or other particulate debris, harvesting the recombinant protein into a cell-free supernatant fraction can optionally involve capture of the recombinant protein by one or more chromatographic capture steps that can partially purify and/or concentrate the protein, such as Protein A or Protein G or Protein L affinity chromatography. (See, e.g., Frank, M. B., “Antibody Binding to Protein A and Protein G beads” 5. In: Frank, M. B., ed., Molecular Biology Protocols. Oklahoma City (1997)).

After harvesting the cell culture fluid comprising a recombinant protein of interest, e.g., an antibody or antibody fragment, can be further purified from the cell-free supernatant fraction. Typically, the purification of recombinant proteins is usually accomplished by an optional series of chromatographic steps such as anion exchange chromatography, cation exchange chromatography, affinity chromatography (using Protein A or Protein G or Protein L as an affinity ligand), hydrophobic interaction chromatography, hydroxy apatite chromatography and size exclusion chromatography. Further, the purification process may comprise one or more ultra-, nano- or diafiltration steps, and/or, optionally, an acidic viral inactivation step. Other optional known techniques for protein purification such as ethanol precipitation, Reverse Phase HPLC, chromatofocusing, SDS-PAGE, and ammonium sulfate precipitation are also possible depending on the antibody to be recovered.

The present method involves harvesting the recombinant antibodies present in the culture supernatant and then purifying the cell-free supernatant fraction by affinity chromatography to purify IgG present in the cell-free supernatant fraction. In this step, affinity chromatography involves loading the cell-free supernatant fraction onto an affinity chromatography matrix having conjugated moieties with particular affinity for immunoglobulin molecules that may be of interest; such conjugated moieties can include, e.g., Protein A, and/or Protein G, and/or Protein L, or anti-kappa antibodies with an affinity for Fab antibody fragments, or anti-his antibodies, or glutathione, or another suitable matrix-conjugated antibody that specifically binds an immunoglobulin epitope of interest. For example, a Protein A matrix can be used to purify proteins that include polypeptides based on human γ1, γ2, or γ4 heavy chains (Lindmark et al., J. Immunol. Meth. 62: 1-13 (1983)). Also useful in the present invention in a Protein A matrix are engineered versions of Protein A that are multimers (typically tetramers, pentamers or hexamers) of a single domain which has been modified to improve its characteristics for industrial applications. “Protein A” is an approximately 42 kDa surface protein originally found in the cell wall of the bacteria Staphylococcus aureus; Protein A is encoded by the spa gene of S. aureus, and its expression in S. aureus is controlled by DNA topology, cellular osmolarity, and a two-component system called ArlS-ArlR. (See, Fournier, B., and Klier, A, Protein A gene expression is regulated by DNA supercoiling which is modified by the ArlS-ArlR two-component system of Staphylococcus aureus, Microbiology 150:3807-19 (2004)). Protein A (Spa gene product) is useful in biochemical research and industry because of its ability to bind immunoglobulins. Protein A is composed of five homologous Ig-binding domains that fold into a three-helix bundle. Each domain is able to bind proteins from many mammalian species, most notably IgGs. It has been shown via crystallographic refinement that the primary binding site for Protein A is on the Fc region, between the C_{H}2 and C_{H}3 domains. (Deisenhofer, J., Crystallographic refinement and atomic models of a human Fc fragment and its complex with fragment B of Protein A from Staphylococcus aureus at 2.9-and 2.8-A resolution, Biochemistry 20 (9): 2361-70 (1981)). In addition, Protein A binds human IgG molecules containing IgG F(ab′)₂ fragments from the human VH3 gene family. (See, Sasso E H, Silverman G J, Mannik M, Human IgA and IgG F(ab′)₂ that bind to staphylococcal Protein A belong to the VHIII subgroup, Journal of Immunology. 147 (6): 1877-83 (1991)). Protein A is typically produced and purified in industrial fermentation for use in immunology, biological research and industrial applications. Natural (or native) Protein A can be cultured in Staphylococcus aureus and contains the five homologous antibody binding regions described above and a C-terminal region for cell wall attachment. Recombinant versions of Protein A, typically produced in Escherichia coli, are also useful for purposes of the invention. For use in the present invention, Protein A matrix can be obtained commercially in various embodiments (e.g., Protein A-Sepharose® from Staphylococcus aureus, from Sigma Aldrich; MabSelect™ Protein A, MabSelect SuRe® Protein A, MabSelect SuRe® LX, and Protein A Sepharose® FF from GE Healthcare Life Sciences; Eshmuno® A Protein A from EMD Millipore; Toyopearl® AF-rProtein A from Tosoh Bioscience; POROS® Protein A from Thermo Fisher Scientific; CaptivA® Protein A affinity resin from Repligen). Recombinant versions of Protein A commonly contain the five homologous antibody binding domains, but for purposes of the present invention can vary in other parts of the structure in order to facilitate covalent coupling to substrates, e.g., resins (such as, but not limited to, agarose). Protein G is recommended for all mouse isotypes and for human γ3 (Guss et al, EMBO J. 5: 15671575 (1986)). Also available commercially (e.g., from Molecular Cloning Laboratories (MCLAB) or Protein Specialists (Prospec)), is recombinant Protein G, an immunoglobulin-binding protein derived from the cell wall of certain strains of beta-hemolytic streptococci. It binds with high affinity to the Fc portion of various classes and subclasses of immunoglobulins from a variety of species. The albumin and cell surface binding domains have been eliminated from Recombinant Protein G to reduce nonspecific binding, although the Fc binding domain is still present and, therefore, can be used to separate IgG from crude samples. The recombinant Protein G is produced in Escherichia coli using sequence from Streptococcus C1-C2-C3. The Protein G contains 200 amino acids (190-384 and five additional residues not including methionine) having a molecular mass of 21.8 kDa. The Protein-G migrates on SDS-PAGE around 32 kDa.

Affinity chromatography matrices containing a combination of Protein A/G are also useful and available commercially. For example, Recombinant Protein A/G fusion protein joins IgG binding domains of both Protein A and Protein G. Recombinant Protein A/G includes four Fc binding domains from Protein A and two from Protein G, yielding a final mass of 50.4 kDa. A version of recombinant Protein A/G consists of 7 IgG-binding domains EDABC-C1C3, which corresponds to the Protein A and G domains that are included in the recombinant sequence. Cell wall binding region, cell membrane binding region and albumin binding region have been removed from the recombinant Protein A/G to ensure specific IgG binding. The Protein A portion is from Staphylococcus aureus segments E, D, A, B and C. The Protein G portion is from Streptococcus segments C1 and C3. The fusion protein has a predicted molecular mass of 47.7 kDa and containing 429 amino acids. The binding dependency to pH of Protein A/G lower than Protein A, but has the additive properties of Protein A and G together. Protein A/G binds to all subclasses of human IgG, making it helpful for purifying polyclonal or monoclonal IgG antibodies whose subclasses have not been identified. Protein L has an affinity for kappa light chains from various species. It can be used to purify monoclonal or polyclonal IgG, IgA, and IgM as well as Fab, F(ab′)2, and recombinant scFv fragments that contain kappa light chains. Protein L is not a universal antibody-binding protein. Protein L binding is restricted to those antibodies that contain kappa light chains. In humans and mice, most antibody molecules contain kappa (κ) light chains and the remainder have lambda (λ) light chains.

Protein L is only effective in binding certain subtypes of kappa light chains. For example, it binds human VκI, VκIII and VκIV subtypes but does not bind the VκII subtype. Binding of mouse immunoglobulins is restricted to those having VκI light chains.

Encompassed within the term “matrix” are resins, beads, nanoparticles, nanofibers, hydrogels, membranes, and monoliths, or any other physical matrix, bearing a relevant covalently bound chromatographic ligand (e.g., Protein A, Protein G, or other affinity chromatographic ligand, such as a target ligand, a charged moiety, or a hydrophobic moiety, etc.) for purposes of the inventive method. The matrix to which the affinity target ligand is attached is most often agarose, but other matrices are available. For example, mechanically stable matrices such as controlled pore glass or poly(styrenedivinyl)benzene allow for faster flow rates and shorter processing times than can be achieved with agarose. Where the protein comprises a CH 3 domain, the Bakerbond ABX™ resin (J. T. Baker, Phillipsburg, N.J.) is useful for purification. An affinity chromatography matrix may be placed or packed into a column useful for the purification of proteins. Loading of the cell-free supernatant fraction onto the affinity chromatography matrix preferably occurs at about neutral pH.

Next, the present method involves loading the mixed pool of IgG molecules from the affinity chromatography step that was generally directed to IgG molecules (e.g., purifying by a Protein A or a Protein G or a Protein L, etc., affinity chromatography matrix) onto a different affinity chromatography matrix, wherein the specific target ligand of interest is covalently conjugated to the affinity chromatography matrix. The affinity matrix with the target ligand covalently attached should have sufficient binding capacity to account for the required mass sufficient to be detected by the mass spectrometer. This can be achieved with either appropriately dense conjugation reactive moieties on the matrix (e.g., resin and/or resin bed size in the column). In producing and storing the conjugated affinity chromatography matrix reagent for future use in this step, the stability of a particular conjugated affinity chromatography matrix needs to be considered might be an issue with regards to the conjugated target ligand itself or the mode by which the ligand is attached to the matrix. Ligand affinity conjugation instability and degradation of the conjugated affinity chromatography matrix reagent during storage can result in decreased antibody yields and/or binding artifacts leading to difficult data analysis or misinterpretation. The practitioner should exercise caution with respect to the appropriate storage conditions and quality control employed to ensure the effective quality of the affinity chromatography matrix before use in the inventive method.

The term “to bind” or “binding” a molecule to an affinity chromatography matrix comprising a covalently-conjugated target moiety, e.g., Protein A or a Protein A matrix, or Protein G or a Protein G matrix, or a particular conjugated target ligand of interest, means exposing the molecule to the affinity chromatography target moiety, under appropriate conditions (e.g., pH and selected salt/buffer composition), such that the molecule is reversibly immobilized in, or on, the affinity chromatography matrix (e.g., a Protein A- or Protein G-conjugated or target ligand-conjugated) by virtue of its binding affinity to the target moiety under those conditions, regardless of the physical mechanism of affinity that may be involved. (See, e.g., Jendeberg, L. et al., The Mechanism of Binding Staphylococcal Protein A to Immunoglobin G Does Not Involve Helix Unwinding, Biochemistry 35(1): 22-31 (1996); Nelson, J. T. et al., Mechanism of Immobilized Protein A Binding to Immunoglobulin G on Nanosensor Array Surfaces, Anal. Chem., 87(16):8186-8193 (2015)).

The inventive high throughput method for selecting an antibody variant amino acid sequence of interest from a plurality of antibody variant amino acid sequences involves a step of eluting the IgG molecules from the affinity chromatography matrix under increasingly stringent buffer conditions and collecting a plurality of eluant fractions.

The term “buffer” or “buffered solution” refers to solutions which resist changes in pH by the action of its conjugate acid-base range. Examples of useful buffers that control pH at ranges of about pH 4 to about pH 8 include phosphate, bicarbonate, acetate, MES, citrate, Tris, bis-tris, histidine, arginine, succinate, citrate, glutamate, and lactate, or a combination of two or more of these, or other mineral acid or organic acid buffers. Salts containing sodium, ammonium, and potassium cations are often used in making a buffered solution.

The term “loading buffer” or “equilibrium buffer” refers to the buffer, and salt or salts, which is mixed with a protein preparation (e.g., a batch or perfusion culture supernatant or filtrate, or an eluant pool containing the antibodies of interest) for loading the protein preparation onto an affinity chromatography matrix, e.g., Protein A- or Protein G-conjugated matrix or a specific target ligand-conjugated affinity chromatography matrix, as the case may be. This buffer is also used to equilibrate the matrix before loading, and to wash after loading the protein.

The term “wash buffer” is used herein to refer to the buffer that is passed over an affinity chromatography matrix, following loading of a protein preparation and prior to elution or after flow-through of the protein of interest. The wash buffer may serve to remove one or more contaminants without substantial elution of the desired protein or can be used to wash out a non-binding protein.

The term “elution buffer” or “eluant” refers to the buffer used to elute the protein of interest (POI) reversibly bound to a matrix. As used herein, the term “solution” refers to either a buffered or a non-buffered solution, including water.

The term “eluting” a molecule (e.g. a desired recombinant protein, such as an antibody of interest, or a contaminant) from an affinity chromatography matrix, means removing the molecule from such material, typically by passing an elution buffer over the affinity chromatography matrix. Eluting a bound protein is typically achieved by increasing the conductivity and/or inducing a pH shift and/or a binding competition. This can be performed either over a linear gradient or a step elution to predetermined conditions. Impurities, particularly HMW species, often bind more tightly than the mAb product and also can be separated from the main desired fraction by adjusting the elution conditions and pool collection criteria (Yigzaw, Y., et al., (2009) supra; Gagnon, P., et al., (1996) supra; Pabst, T. M., et al., (2009) Journal of Chromatography 1216, 7950-7956). The molecular interaction under consideration dictates the type of elution methods that can be used. Thus, salt can be used to disrupt hydrophobic interactions whereas pH can disrupt ionic and hydrogen binds. Other elution methods besides ionic strength and pH can be used to disrupt the interaction between the antibody and ligand. A peptide specific for the antibody epitope on the target ligand can be used to compete with the on-rate and affinity binding properties of the antibody. A small organic molecule can be used in a similar fashion as a peptide. As part of the screening process for a viable candidate, stress can be applied to the antibody pool prior to binding to the ligand affinity column. Thermal, chemical and/or pH stress can induce a conformational change or denaturation event resulting in aggregation of the antibody which can be removed via precipitation (centrifugation or ultrafiltration) or preparative SEC. This step will remove non-viable candidates from binding to the target affinity matrix. Furthermore, the stress can lead to non-aggregated, non-native antibody material which will have decreased binding affinity to the target resulting in selecting against these poor binders. This can simplify the interpretation of the screening data obtained from employing the inventive method.

The phrase “increasingly stringent buffer conditions” means employing a gradient (a step gradient or a linear gradient) of an increasingly more challenging condition by which antibody variants can be distinguished from each other. Examples include, but are not limited to, a gradient (a step gradient or a linear gradient) of increasing ionic strength (typically with higher conductivity going up to about 40-150 mS), or a pH gradient (a step gradient or a linear gradient) approaching an extreme of lower or higher pH than the initial buffer condition, or a gradient (a step gradient or a linear gradient) of increasing concentration of a molecule that competes for binding to the target ligand, such as but limited to, a small molecule or an oligopeptide.

The term “elution pool” or “eluant pool” means the material eluted from a chromatography matrix, which material includes the recombinant protein of interest, e.g., an antibody of interest.

The term “loading,” with respect to an affinity chromatography matrix, means loading a protein preparation (e.g., a batch or perfusion culture supernatant or filtrate, or an eluant pool containing the protein of interest) onto the affinity chromatography matrix.

The term “washing,” with respect to an affinity chromatography matrix, means passing an appropriate buffer through or over the affinity chromatography matrix.

“Under physiological conditions” with respect to incubating buffers and immunoglobulins, or other binding assay reagents means incubation under conditions of temperature, pH, and ionic strength, that permit a biochemical reaction, such as a non-covalent binding reaction, to occur. Typically, the temperature is at room or ambient temperature up to about 37° C. and at pH 6.5-7.5.

“Physiologically acceptable salt” of a composition of matter, for example a salt of a protein of interest, e.g., a fusion protein, or another immunoglobulin, such as an antibody, or any other protein of interest, or a salt of an amino acid, such as, but not limited to, a lysine, histidine, or proline salt, means any salt, or salts, that are known or later discovered to be pharmaceutically acceptable. Some non-limiting examples of pharmaceutically acceptable salts are: Some non-limiting examples of pharmaceutically acceptable salts are: acetate salts; trifluoroacetate salts; hydrohalides, such as hydrochloride (e.g., monohydrochloride or dihydrochloride salts) and hydrobromide salts; sulfate salts; citrate salts; maleate salts; tartrate salts; glycolate salts; gluconate salts; succinate salts; mesylate salts; besylate salts; salts of gallic acid esters (gallic acid is also known as 3,4, 5 trihydroxybenzoic acid) such as pentagalloylglucose (PGG) and epigallocatechin gallate (EGCG), salts of cholesteryl sulfate, pamoate salts, tannate salts, and oxalate salts.

A “reaction mixture” is an aqueous mixture containing all the reagents and factors necessary, which under physiological conditions of incubation, permit an in vitro biochemical reaction of interest to occur, such as a covalent or non-covalent binding reaction.

A “domain” or “region” (used interchangeably herein) of a polynucleotide is any portion of the entire polynucleotide, up to and including the complete polynucleotide, but typically comprising less than the complete polynucleotide. A domain can, but need not, fold independently (e.g., DNA hairpin folding) of the rest of the polynucleotide chain and/or be correlated with a particular biological, biochemical, or structural function or location, such as a coding region or a regulatory region.

A “domain” or “region” (used interchangeably herein) of a protein is any portion of the entire protein, up to and including the complete protein, but typically comprising less than the complete protein. A domain can, but need not, fold independently of the rest of the protein chain and/or be correlated with a particular biological, biochemical, or structural function or location (e.g., a ligand binding domain, or a cytosolic, transmembrane or extracellular domain).

Quantification of immunoglobulin protein (e.g., an antibody), is often useful or necessary in tracking protein. An antibody that specifically binds a domain of the antibody or antibodies of interest, particularly a specific monoclonal antibody, can therefore be useful for these purposes.

The term “antibody”, or interchangeably “Ab”, is used in the broadest sense and includes fully assembled antibodies, monoclonal antibodies (including human, humanized or chimeric antibodies), polyclonal antibodies, multispecific antibodies (e.g., bispecific antibodies), and antibody fragments that can bind antigen (e.g., Fab, Fab′, F(ab′)₂, Fv, single chain antibodies, diabodies), comprising complementarity determining regions (CDRs) of the foregoing as long as they exhibit the desired biological activity. Multimers or aggregates of intact molecules and/or fragments, including chemically derivatized antibodies, are contemplated. Antibodies of any isotype class or subclass, including IgG, IgM, IgD, IgA, and IgE, IgG1, IgG2, IgG3, IgG4, IgA1 and IgA2, or any allotype, are contemplated. Different isotypes have different effector functions; for example, IgG1 and IgG3 isotypes have antibody-dependent cellular cytotoxicity (ADCC) activity.

An “isolated” protein, e.g., an antibody protein, is one that has been identified and separated from one or more components of its natural environment or of a culture medium in which it has been secreted by a producing cell. In some embodiments, the isolated protein is substantially free from proteins or polypeptides or other contaminants that are found in its natural or culture medium environment that would interfere with its therapeutic, diagnostic, prophylactic, research or other use. “Contaminant” components of its natural environment or medium are materials that would interfere with diagnostic or therapeutic uses for the protein, e.g., an antibody, and may include enzymes, hormones, and other proteinaceous or nonproteinaceous (e.g., polynucleotides, lipids, carbohydrates) solutes. Typically, an “isolated protein” constitutes at least about 5%, at least about 10%, at least about 25%, or at least about 50% of a given sample. In some embodiments, the protein of interest, e.g., an antibody, will be purified (1) to greater than 95% by weight of protein, and most preferably more than 99% by weight, or (2) to homogeneity by SDS-PAGE, or other suitable technique, under reducing or nonreducing conditions, optionally using a stain, e.g., Coomassie blue or silver stain. Isolated naturally occurring antibody includes the antibody in situ within recombinant cells since at least one component of the protein's natural environment will not be present. Typically, however, the isolated protein of interest (e.g., an antibody) will be prepared by at least one purification step.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies that are antigen binding proteins are highly specific binders, being directed against an individual antigenic site or epitope, in contrast to polyclonal antibody preparations that typically include different antibodies directed against different epitopes. Nonlimiting examples of monoclonal antibodies include murine, rabbit, rat, chicken, chimeric, humanized, or human antibodies, fully assembled antibodies, multispecific antibodies (including bispecific antibodies), antibody fragments that can bind an antigen (including, Fab, Fab′, F(ab)₂, Fv, single chain antibodies, diabodies), maxibodies, nanobodies, and recombinant peptides comprising CDRs of the foregoing as long as they exhibit the desired biological activity, or variants or derivatives thereof.

The modifier “monoclonal” indicates the character of the antibody as being obtained from a substantially homogeneous population of antibodies, and is not to be construed as requiring production of the antibody by any particular method. For example, monoclonal antibodies may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The term “immunoglobulin” encompasses full antibodies comprising two dimerized heavy chains (HC), each covalently linked to a light chain (LC); a single undimerized immunoglobulin heavy chain and covalently linked light chain (HC+LC), or a chimeric immunoglobulin (light chain+heavy chain)-Fc heterotrimer (a so-called “hemibody”). An “immunoglobulin” is a protein, but is not necessarily an antigen binding protein.

In an “antibody”, each tetramer is composed of two identical pairs of polypeptide chains, each pair having one “light” chain of about 220 amino acids (about 25 kDa) and one “heavy” chain of about 440 amino acids (about 50-70 kDa). The amino-terminal portion of each chain includes a “variable” (“V”) region of about 100 to 110 or more amino acids primarily responsible for antigen recognition. The carboxy-terminal portion of each chain defines a constant region primarily responsible for effector function. The variable region differs among different antibodies. The constant region is the same among different antibodies. Within the variable region of each heavy or light chain, there are three hypervariable subregions that help determine the antibody's specificity for antigen in the case of an antibody that is an antigen binding protein. The variable domain residues between the hypervariable regions are called the framework residues and generally are somewhat homologous among different antibodies Immunoglobulins can be assigned to different classes depending on the amino acid sequence of the constant domain of their heavy chains. Human light chains are classified as kappa (.kappa.) and lambda (.lamda.) light chains. Within light and heavy chains, the variable and constant regions are joined by a “J” region of about 12 or more amino acids, with the heavy chain also including a “D” region of about 10 more amino acids. See generally, Fundamental Immunology, Ch. 7 (Paul, W., ed., 2nd ed. Raven Press, N.Y. (1989)). An “antibody” also encompasses a recombinantly made antibody, and antibodies that are glycosylated or lacking glycosylation.

The term “light chain” or “immunoglobulin light chain” includes a full-length light chain and fragments thereof having sufficient variable region sequence to confer binding specificity. A full-length light chain includes a variable region domain, V_{L}, and a constant region domain, C_{L}. The variable region domain of the light chain is at the amino-terminus of the polypeptide. Light chains include kappa chains and lambda chains.

The term “heavy chain” or “immunoglobulin heavy chain” includes a full-length heavy chain and fragments thereof having sufficient variable region sequence to confer binding specificity. A full-length heavy chain includes a variable region domain, V_{H}, and three constant region domains, C_{H1}, C_{H2}, and C_{H3}. The V_{H} domain is at the amino-terminus of the polypeptide, and the C_{H} domains are at the carboxyl-terminus, with the C_{H3} being closest to the carboxy-terminus of the polypeptide. Heavy chains are classified as mu (μ), delta (δ), gamma (γ), alpha (α), and epsilon (ε), and define the antibody's isotype as IgM, IgD, IgG, IgA, and IgE, respectively. Heavy chains may be of any isotype, including IgG (including IgG1, IgG2, IgG3 and IgG4 subtypes), IgA (including IgA1 and IgA2 subtypes), IgM and IgE. Several of these may be further divided into subclasses or isotypes, e.g. IgG1, IgG2, IgG3, IgG4, IgA1 and IgA2. Different IgG isotypes may have different effector functions (mediated by the Fc region), such as antibody-dependent cellular cytotoxicity (ADCC) and complement-dependent cytotoxicity (CDC). In ADCC, the Fc region of an antibody binds to Fc receptors (Fc.gamma.Rs) on the surface of immune effector cells such as natural killers and macrophages, leading to the phagocytosis or lysis of the targeted cells. In CDC, the antibodies kill the targeted cells by triggering the complement cascade at the cell surface.

An “Fc region”, or used interchangeably herein, “Fc domain” or “immunoglobulin Fc domain”, contains two heavy chain fragments, which in a full antibody comprise the C_{H1} and C_{H2} domains of the antibody. The two heavy chain fragments are held together by two or more disulfide bonds and by hydrophobic interactions of the C_{H3} domains.

The term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

For a detailed description of the structure and generation of antibodies, see Roth, D. B., and Craig, N. L., Cell, 94:411-414 (1998), herein incorporated by reference in its entirety. Briefly, the process for generating DNA encoding the heavy and light chain immunoglobulin sequences occurs primarily in developing B-cells. Prior to the rearranging and joining of various immunoglobulin gene segments, the V, D, J and constant (C) gene segments are found generally in relatively close proximity on a single chromosome. During B-cell-differentiation, one of each of the appropriate family members of the V, D, J (or only V and J in the case of light chain genes) gene segments are recombined to form functionally rearranged variable regions of the heavy and light immunoglobulin genes. This gene segment rearrangement process appears to be sequential. First, heavy chain D-to-J joints are made, followed by heavy chain V-to-DJ joints and light chain V-to-J joints. In addition to the rearrangement of V, D and J segments, further diversity is generated in the primary repertoire of immunoglobulin heavy and light chains by way of variable recombination at the locations where the V and J segments in the light chain are joined and where the D and J segments of the heavy chain are joined. Such variation in the light chain typically occurs within the last codon of the V gene segment and the first codon of the J segment. Similar imprecision in joining occurs on the heavy chain chromosome between the D and J_{H} segments and may extend over as many as 10 nucleotides. Furthermore, several nucleotides may be inserted between the D and J_{H} and between the V_{H} and D gene segments which are not encoded by genomic DNA. The addition of these nucleotides is known as N-region diversity. The net effect of such rearrangements in the variable region gene segments and the variable recombination which may occur during such joining is the production of a primary antibody repertoire.

The term “hypervariable” region refers to the amino acid residues of an antibody which are responsible for antigen-binding. The hypervariable region comprises amino acid residues from a complementarity determining region or CDR [i.e., residues 24-34 (L1), 50-56 (L2) and 89-97 (L3) in the light chain variable domain and 31-35 (H1), 50-65 (H2) and 95-102 (H3) in the heavy chain variable domain as described by Kabat et al., Sequences of Proteins of Immunological Interest, th Ed. Public Health Service, National Institutes of Health, Bethesda, Md. (1991)]. Even a single CDR may recognize and bind antigen, although with a lower affinity than the entire antigen binding site containing all of the CDRs.

An alternative definition of residues from a hypervariable “loop” is described by Chothia et al., J. Mol. Biol. 196: 901-917 (1987) as residues 26-32 (L1), 50-52 (L2) and 91-96 (L3) in the light chain variable domain and 26-32 (H1), 53-55 (H2) and 96-101 (H3) in the heavy chain variable domain.

“Framework” or “FR” residues are those variable region residues other than the hypervariable region residues.

“Antibody fragments” comprise a portion of an intact full length antibody, preferably the antigen binding or variable region of the intact antibody. Examples of antibody fragments include Fab, Fab′, F(ab′)₂, and Fv fragments; diabodies; linear antibodies (Zapata et al., Protein Eng., 8(10):1057-1062 (1995)); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment which contains the constant region. The Fab fragment contains all of the variable domain, as well as the constant domain of the light chain and the first constant domain (CH1) of the heavy chain. The Fc fragment displays carbohydrates and is responsible for many antibody effector functions (such as binding complement and cell receptors), that distinguish one class of antibody from another.

Pepsin treatment yields an F(ab′)₂ fragment that has two “Single-chain Fv” or “scFv” antibody fragments comprising the V_{H} and V_{L} domains of antibody, wherein these domains are present in a single polypeptide chain. Fab fragments differ from Fab′ fragments by the inclusion of a few additional residues at the carboxy terminus of the heavy chain CH1 domain including one or more cysteines from the antibody hinge region. Preferably, the Fv polypeptide further comprises a polypeptide linker between the VH and VL domains that enables the Fv to form the desired structure for antigen binding. For a review of scFv see Pluckthun in The Pharmacology of Monoclonal Antibodies, vol. 1 13, Rosenburg and Moore eds., Springer-Verlag, New York, pp. 269-315 (1994).

A “Fab fragment” is comprised of one light chain and the C_{H1} and variable regions of one heavy chain. The heavy chain of a Fab molecule cannot form a disulfide bond with another heavy chain molecule.

A “Fab′ fragment” contains one light chain and a portion of one heavy chain that contains the V_{H} domain and the C_{H1} domain and also the region between the C_{H1} and C_{H2} domains, such that an interchain disulfide bond can be formed between the two heavy chains of two Fab′ fragments to form an F(ab′)₂ molecule.

A “F(ab′)₂ fragment” contains two light chains and two heavy chains containing a portion of the constant region between the C_{H1} and C_{H2} domains, such that an interchain disulfide bond is formed between the two heavy chains. A F(ab′)₂ fragment thus is composed of two Fab′ fragments that are held together by a disulfide bond between the two heavy chains.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen binding site on the surface of the VH VL dimer. A single variable domain (or half of an Fv comprising only three CDRs specific for an antigen) has the ability to recognize and bind antigen, although at a lower affinity than the entire binding site.

“Single-chain antibodies” are Fv molecules in which the heavy and light chain variable regions have been connected by a flexible linker to form a single polypeptide chain, which forms an antigen-binding region. Single chain antibodies are discussed in detail in International Patent Application Publication No. WO 88/01649 and U.S. Pat. Nos. 4,946,778 and 5,260,203, the disclosures of which are incorporated by reference in their entireties.

“Single-chain Fv” or “scFv” antibody fragments comprise the V_{H} and V_{L} domains of antibody, wherein these domains are present in a single polypeptide chain, and optionally comprising a polypeptide linker between the V_{H} and V_{L} domains that enables the Fv to form the desired structure for antigen binding (Bird et al., Science 242:423-426, 1988, and Huston et al., Proc. Nati. Acad. Sci. USA 85:5879-5883, 1988). An “Fd” fragment consists of the V_{H} and C_{H1} domains.

The term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

A “domain antibody” is an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

The term “antigen binding protein” (ABP) includes antibodies or antibody fragments, as defined herein, that specifically bind a target ligand or antigen of interest.

In general, an antigen binding protein, e.g., an immunoglobulin protein, or an antibody or antibody fragment, “specifically binds” to a target ligand or antigen of interest when it has a significantly higher binding affinity for, and consequently is capable of distinguishing, that target ligand or antigen, compared to its affinity for other unrelated proteins, under similar binding assay conditions. Typically, an antigen binding protein is said to “specifically bind” its target antigen when the dissociation constant (K_{D}) is 10⁻⁸ M or lower. The antigen binding protein specifically binds antigen with “high affinity” when the K_{D} is 10⁻⁹ M or lower, and with “very high affinity” when the K_{D} is 10⁻¹⁰ M or lower.

“Antigen binding region” or “antigen binding site” means a portion of a protein that specifically binds a specified target ligand or antigen. For example, that portion of an antigen binding protein that contains the amino acid residues that interact with a target ligand or an antigen and confer on the antigen binding protein its specificity and affinity for the antigen is referred to as “antigen binding region.” In an antibody, an antigen binding region typically includes one or more “complementary binding regions” (“CDRs”). Certain antigen binding regions also include one or more “framework” regions (“FRs”). A “CDR” is an amino acid sequence that contributes to antigen binding specificity and affinity. “Framework” regions can aid in maintaining the proper conformation of the CDRs to promote binding between the antigen binding region and an antigen. In a traditional antibody, the CDRs are embedded within a framework in the heavy and light chain variable region where they constitute the regions responsible for antigen binding and recognition. A variable region of an immunoglobulin antigen binding protein comprises at least three heavy or light chain CDRs, see, supra (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991, supra; see also Chothia and Lesk, 1987, supra).

The term “target” or “antigen” refers to a molecule or a portion of a molecule capable of being bound by a selective binding agent, such as an antigen binding protein (including, e.g., an antibody or immunologically functional fragment of an antibody), and additionally capable of being used in an animal to produce antibodies capable of binding to that antigen. An antigen may possess one or more epitopes that are capable of interacting with different antigen binding proteins, e.g., antibodies.

The term “epitope” is the portion of a target molecule that is bound by an antigen binding protein (for example, an antibody or antibody fragment). The term includes any determinant capable of specifically binding to an antigen binding protein, such as an antibody or to a T-cell receptor. An epitope can be contiguous or non-contiguous (e.g., in a single-chain polypeptide, amino acid residues that are not contiguous to one another in the polypeptide sequence but that within the context of the molecule are bound by the antigen binding protein). In certain embodiments, epitopes may be mimetic in that they comprise a three dimensional structure that is similar to an epitope used to generate the antigen binding protein, yet comprise none or only some of the amino acid residues found in that epitope used to generate the antigen binding protein. Most often, epitopes reside on proteins, but in some instances may reside on other kinds of molecules, such as nucleic acids. Epitope determinants may include chemically active surface groupings of molecules such as amino acids, sugar side chains, phosphoryl or sulfonyl groups, and may have specific three dimensional structural characteristics, and/or specific charge characteristics. Generally, antigen binding proteins specific for a particular target will preferentially recognize an epitope on the target in a complex mixture of proteins and/or macromolecules.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, Wis.). The computer algorithm GAP is used to align the two polypeptides or two polynucleotides for which the percent sequence identity is to be determined. The sequences are aligned for optimal matching of their respective amino acid or nucleotide (the “matched span”, as determined by the algorithm). A gap opening penalty (which is calculated as 3.times. the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptides or nucleotide sequences using the GAP program include the following:

Algorithm: Needleman et al., 1970, J. Mol. Biol. 48:443-453;

Comparison matrix: BLOSUM 62 from Henikoff et al., 1992, supra;

Gap Penalty: 12 (but with no penalty for end gaps)

Gap Length Penalty: 4

Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The term “modification” when used in connection with proteins of interest, include, but are not limited to, one or more amino acid changes (including substitutions, insertions or deletions); chemical modifications; covalent modification by conjugation to therapeutic or diagnostic agents; labeling (e.g., with radionuclides or various enzymes); covalent polymer attachment such as PEGylation (derivatization with polyethylene glycol) and insertion or substitution by chemical synthesis of non-natural amino acids. By methods known to the skilled artisan, proteins, can be “engineered” or modified for improved target affinity, selectivity, stability, and/or manufacturability before the coding sequence of the “engineered” protein is included in the expression cassette.

The term “derivative,” when used in connection with proteins of interest, refers to proteins that are covalently modified by conjugation to therapeutic or diagnostic agents, labeling (e.g., with radionuclides or various enzymes), covalent polymer attachment such as PEGylation (derivatization with polyethylene glycol) and insertion or substitution of natural or non-natural amino acids.

Cloning DNA

Cloning of DNA is carried out using standard techniques (see, e.g., Sambrook et al. (1989) Molecular Cloning: A Laboratory Guide, Vols 1-3, Cold Spring Harbor Press, which is incorporated herein by reference). For example, a cDNA library may be constructed by reverse transcription of polyA+ mRNA, preferably membrane-associated mRNA, and the library screened using probes specific for human immunoglobulin polypeptide gene sequences. In one embodiment, however, the polymerase chain reaction (PCR) is used to amplify cDNAs (or portions of full-length cDNAs) encoding an immunoglobulin gene segment of interest (e.g., a light or heavy chain variable segment). The amplified sequences can be readily cloned into any suitable vector, e.g., expression vectors, minigene vectors, or phage display vectors. It will be appreciated that the particular method of cloning used is not critical, so long as it is possible to determine the sequence of some portion of the polypeptide of interest, e.g., antibody sequences.

One source for antibody nucleic acids is a hybridoma produced by obtaining a B cell from an animal immunized with the antigen of interest and fusing it to an immortal cell. Alternatively, nucleic acid can be isolated from B cells (or whole spleen) of the immunized animal. Yet another source of nucleic acids encoding antibodies is a library of such nucleic acids generated, for example, through phage display technology. Polynucleotides encoding peptides of interest, e.g., variable region peptides with desired binding characteristics, can be identified by standard techniques such as panning.

Sequencing of DNA is carried out using standard techniques (see, e.g., Sambrook et al. (1989) Molecular Cloning: A Laboratory Guide, Vols 1-3, Cold Spring Harbor Press, and Sanger, F. et al. (1977) Proc. Natl. Acad. Sci. USA 74: 5463-5467, which is incorporated herein by reference). By comparing the sequence of the cloned nucleic acid with published sequences of genes and cDNAs, one of skill will readily be able to determine, depending on the region sequenced. One source of gene sequence information is the National Center for Biotechnology Information, National Library of Medicine, National Institutes of Health, Bethesda, Md.

In accordance with the present high throughput method for selecting an antibody variant amino acid sequence of interest from a plurality of antibody variant amino acid sequences, so-called “Next-generation” sequencing is a preferred method for confirming the presence of all engineered DNA constructs prior to the transfection step. (See, e.g., Buermans, H. P. J., & den Dunnen, J. T., Next generation sequencing technology: Advances and applications, Biochimica et Biophysica Acta—Molecular Basis of Disease 1842(10): 1932-1941 (2014)). Sanger will provide an indication of the species present but not as individual designs. Sequencing will validate that the absence of any species was not due to their absence as a DNA construct. There is a possibility that some engineered designs will not be expressed and secreted at high enough levels to survive all processing steps and be detected by mass spectrometry. This may result because certain engineered antibody variant designs are unstable, but such variants will not likely be viable as therapeutics anyway. This can be viewed as part of the screening process, however, since typically it is desirable to find antibody variant candidates that do express well for manufacturing purposes.

Chemical synthesis of parts or the whole of a coding region containing codons reflecting desires protein changes can be cloned into an expression vector by either restriction digest and ligation of 5′ and 3′ ends of fragments or the entire open reading frame (ORF), containing nucleotide overhangs that are generated by restriction enzyme digestion and which are compatible to the destination vector. The fragments or inserts are typically ligated into the destination vector using a T4 ligase or other common enzyme. Other useful methods are similar to the above except that the cut site for the restriction enzyme is at location different from the recognition sequence. Alternatively, isothermal assembly (i.e., “Gibson Assembly”) can be employed, in which nucleotide overhangs are generated during synthesis of fragments or ORFs; digestion by exonucleases is employed. Alternatively, nucleotide overhangs can be ligated ex vivo by a ligase or polymerase or in vivo by intracellular processes.

Alternatively, homologous recombination can be employed, similar to isothermal assembly, except exonuclease activity of T4 DNA ligase can used on both insert and vector and ligation can be performed in vivo.

Another useful cloning method is the so-called “TOPO” method, in which a complete insert containing a 3′ adenosine overhang (generated by Taq polymerase) is present, and Topoisomerase I ligates the insert into a TOPO vector.

Another useful cloning method is degenerate or error-prone PCR exploiting degenerate primers and/or a thermally stable low-fidelity polymerase caused by the polymerase within certain reaction conditions. Fragments or inserts are then cloned into an expression vector.

The above are merely examples of known cloning techniques, and the skilled practitioner knows how to employ any other suitable cloning techniques.

Isolated DNA can be operably linked to control sequences or placed into expression vectors, which are then transfected into host cells that do not otherwise produce immunoglobulin protein, to direct the synthesis of monoclonal antibodies in the recombinant host cells. Recombinant production of antibodies is well known in the art.

Nucleic acid is operably linked when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

Many vectors are known in the art. Vector components may include one or more of the following: a signal sequence (that may, for example, direct secretion of the expressed protein by the recombinant host cells); an origin of replication, one or more selective marker genes (that may, for example, confer antibiotic or other drug resistance, complement auxotrophic deficiencies, or supply critical nutrients not available in the media), an enhancer element, a promoter, and a transcription termination sequence, all of which are well known in the art.

Antibody Expression

The present high throughput method for selecting an antibody variant amino acid sequence of interest from a plurality of antibody variant amino acid sequences involves the step of transfecting a plurality of mammalian cells with the mixed pool of nucleic acids from the cloning step. Transfecting can be by transient or stable transfection, e.g., the pooled plasmid constructs (expression vectors) from the cloning step can be transfected into a plurality of host cells (e.g., mammalian, e.g., HEK 293 or CHO, bacterial, insect, yeast cells) for expression using a cationic lipid, polyethylenimine, Lipofectamine™, or ExpiFectamine™, or electroporation. The skilled practitioner is aware of numerous suitable means for transfecting to achieve expression of recombinant antibodies.

The present high throughput method for selecting an antibody variant amino acid sequence of interest from a plurality of antibody variant amino acid sequences involves the step of culturing the transfected mammalian cells under physiological conditions allowing the cells to express recombinant antibodies. Most conveniently, the expressed recombinant antibodies are directly secreted into the culture supernatant (by employing appropriate secretory-directing signal peptides) and are harvested therefrom; otherwise additional steps will be needed to isolate the expressed antibodies from a cell extract.

For purposes of the present invention, the desired scale of the recombinant expression will be dependent on the type of expression system and the quantity of different theoretical antibody variants to be studied. Some expression systems such as ExpiCHO™ usually produce higher yields as compared to some earlier HEK293 technologies. A smaller scale ExpiCHO™ might then suffice as compared to an HEK293 system. Efficiency of variant pool transfection can also be a consideration in choosing an appropriate expression system. Electroporation can be a suitable method given its effectiveness, relative low cost and the fact that high-throughput during this step is not critical. Additionally, the ratio of light chain to heavy chain can be varied during the co-transfection to improve expression of certain variants. The different LC-HC co-expressions can either be pooled prior to Protein A or Protein G affinity purification, or kept separate through the entire process. Pooling the products allows for easier processing but keeping them separate can provide relevant information during manufacturing scale-up, since it would be known that particular antibodies that were selected employed different LC-HC ratios. The product yield for a given variant has to be sufficient to survive numerous handling steps and produce a signal high enough to be detected by the chosen mass spectrometry system. The sensitivity of currently available mass spectrometers allows for the detection of sub-microgram amounts of antibody in a typical injection volume of 1 μg. Even accounting for purification loss at both steps of affinity chromatography (using, e.g., Protein A, to isolate or obtain a mixed pool of IgG molecules, and then a specific target ligand), and buffer exchanges and multiple fractions this means milligram expression levels of antibody typically suffice. One must keep in mind that the inventive method involves a binding competition of all of the engineered variants in the mixed pool of IgG molecules. Some low IgG expressers might be tighter binders to the target ligand but if they are outcompeted by a larger molar quantity of IgG variants with weaker binding to the target, they may not be detected. The higher the level of the potentially desirable variants the higher the selection stringency can be and yet still be detected by the mass spectrometer. Typically, 100 milligrams of total antibody protein will suffice, requiring only a cell culture batch of 20 mL to 500 mL; while larger scale culture batches or continuous cell culture methods can be employed, larger volumes are typically not cost-effective.

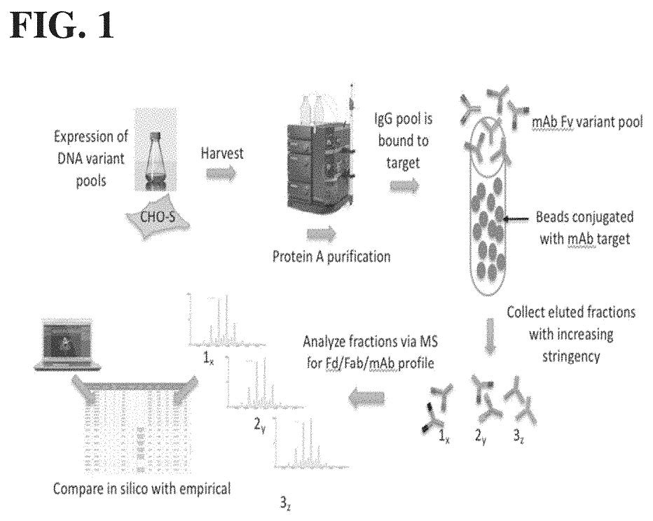

One embodiment of the inventive high throughput method for selecting an antibody variant amino acid sequence of interest from a plurality of antibody variant amino acid sequences is shown schematically in FIG. 1.

By way of further illustration, the following numbered embodiments are encompassed by the present invention:

### Embodiment 1

A high throughput method for selecting an antibody variant amino acid sequence of interest from a plurality of antibody variant amino acid sequences, comprising the steps of:

(a) predetermining a plurality of variant amino acid sequences and the corresponding molecular weight of each member of the plurality of variant amino acid sequences, wherein the variant amino acid sequences are variants of a preselected reference antibody, wherein the parent antibody specifically binds to a target ligand of interest;

(b) cloning a plurality of nucleic acid sequences, each encoding a member of the plurality of variant amino acid sequences, to generate a mixed pool of nucleic acids capable of transfecting a mammalian cell;

(c) transfecting a plurality of mammalian cells with the mixed pool of nucleic acids from step (b);

(d) culturing the transfected mammalian cells under physiological conditions allowing the cells to express recombinant antibodies;

(e) harvesting the recombinant antibodies present in the culture in step (d) into a cell-free supernatant fraction and purifying the cell-free supernatant fraction by affinity chromatography to obtain a mixed pool of IgG molecules;

(f) loading the mixed pool of IgG molecules from step (e) onto an affinity chromatography matrix, wherein the target ligand of interest is covalently conjugated to the affinity chromatography matrix;

(g) eluting the IgG molecules from the affinity chromatography matrix under increasingly stringent buffer conditions and collecting a plurality of eluant fractions; and

(h) detecting the molecular weights of the IgG molecules present in each eluant fraction by mass spectrometry,

whereby one or more antibody variants of interest, from the eluant fraction obtained under the highest stringency buffer conditions in step (g), having a predetermined variant amino acid sequence, is identified by its corresponding molecular weight and can be selected from the plurality of variant amino acid sequences for further analysis.

### Embodiment 2

The method of Embodiment 1, wherein culturing the transfected mammalian cells under physiological conditions allowing the cells to express recombinant antibodies comprises conditions allowing the cells to secrete the recombinant antibodies into the culture supernatant.

### Embodiment 3

The method of Embodiments 1-2, wherein eluting the IgG molecules from the affinity chromatography matrix under increasingly stringent buffer conditions comprises employing a gradient of increasing ionic strength.

### Embodiment 4

The method of Embodiments 1-3, wherein eluting the IgG molecules from the affinity chromatography matrix under increasingly stringent buffer conditions comprises employing a pH gradient.

### Embodiment 5

The method of Embodiments 1-4, wherein eluting the IgG molecules from the affinity chromatography matrix under increasingly stringent buffer conditions comprises employing a gradient of increasing concentration of a molecule that competes for binding to the target ligand.

### Embodiment 6

The method of Embodiments 1-5, wherein the molecule that competes for binding to the target ligand is a small molecule or an oligopeptide.

The following working examples are illustrative and not to be construed in any way as limiting the scope of the invention.

### EXAMPLES

### Example 1. Materials and Methods

Pre-Selecting Parental Antibody.

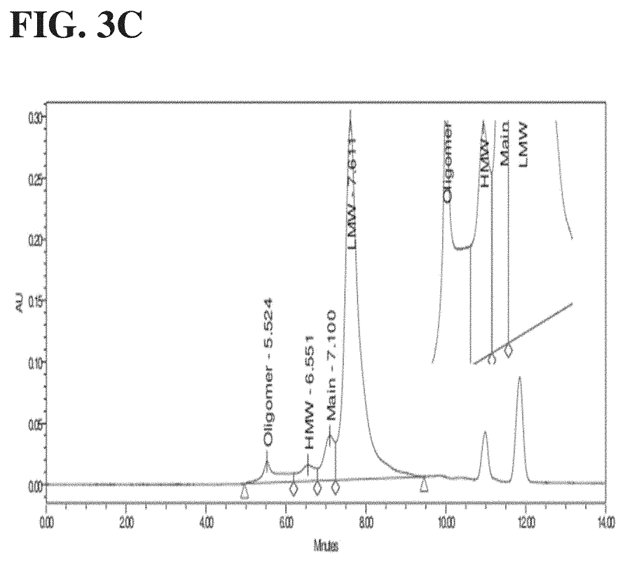

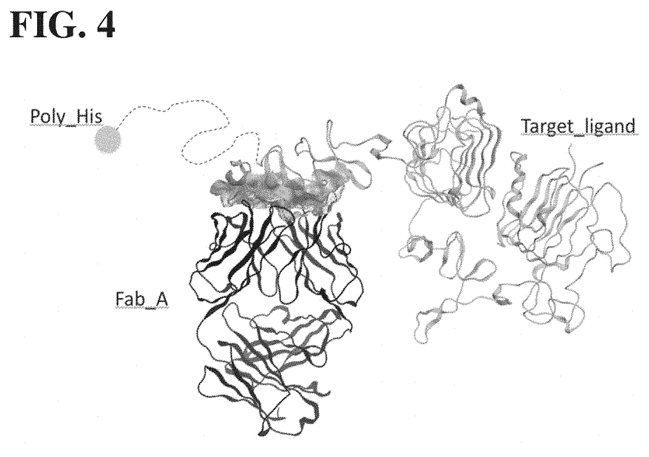

A well studied antibody/ligand interaction system was chosen to test and demonstrate the effectiveness of the inventive high throughput method for selecting an antibody variant amino acid sequence of interest from a plurality of antibody variant amino acid sequences. The antibody-target interaction between trastuzumab (hereinafter referred to as “mAb_A”) and ErbB2 (hereinafter referred to as “ligand”) was preselected since this mAb_A/ligand system is well studied, making it possible to confer with the existing scientific literature. Both this preselected parental antibody (i.e., mAb_A) and the ligand are easily expressed and downstream handling is manageable. The mAb/ligand structure has been solved and it can be a useful tool in engineering design and data analysis (See, FIG. 4). The RCSB Protein Data Bank (PDB) file 1N8Z (available at rcsb.org/structure/1N8Z), was downloaded to determine suitable placement for a polyHis tag on the ligand protein and also to enable us to compare our findings from our mAb_A variant screening to the published ligand-bound structure. We experimentally corroborated the published literature to validate the tag placement. Although, electron density was absent from the C-terminus of the ligand, the evidence in the literature (Kanthala, S. et al., Expression and Purification of HER2 Extracellular Domain Proteins, In: Schneider 2 Insect Cells. Protein Expression and Purification, 125(26-33):1-21 (2016)) indicated that the C-terminus was an appropriate location for conjugating the polyHis tag (FIG. 4).

For some parent antibodies of interest, a high-resolution antibody-ligand structure might not be available for a given screening project, as they are for the mAb_A/ligand system employed here. In such a case, the purification tag (e.g., polyHis) can then be engineered on either end of the appropriate ligand without any knowledge of the binding epitope. However, it is possible that either the N-terminus or C-terminus could be within the binding epitope, and therefore another purification strategy can be used, such as cleaving off the purification tag prior to generation of the conjugated affinity chromatography resin. Before proceeding to affinity chromatography resin generation, a simple antibody-ligand binding experiment such as an ELISA allows confirmation that the epitope is still accessible and conformationally unperturbed. To simplify the process the purification of the ligand and generation of the affinity resin can be one-step. For instance, after employing the purification tag (e.g., polyHis, GST, MBP, FLAG, or other suitable purification tag) for selection of the target species, the bound ligand can be stabilized post-washing via covalent crosslinking to the matrix or other functional group (e.g., a functional moiety of a protein, DNA, or carbohydrate) that is already bound to the resin. However, there is a risk with this strategy that the target ligand orientation might be inappropriate for antibody binding, if the epitope is concealed.

Predetermining Variants and Cloning.

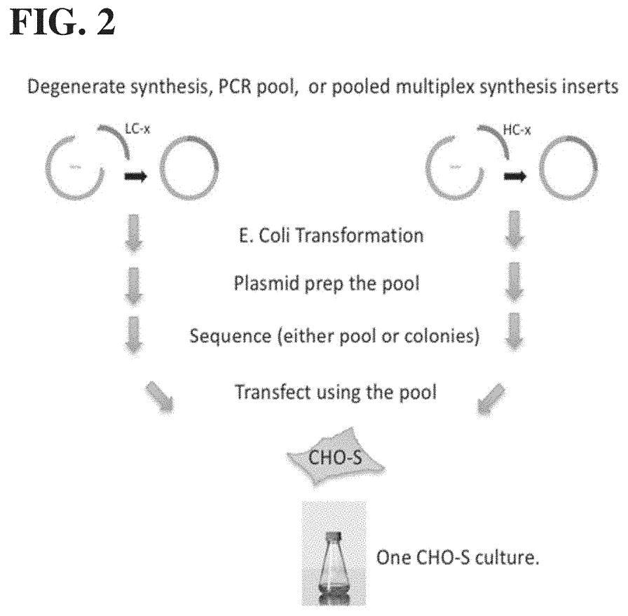

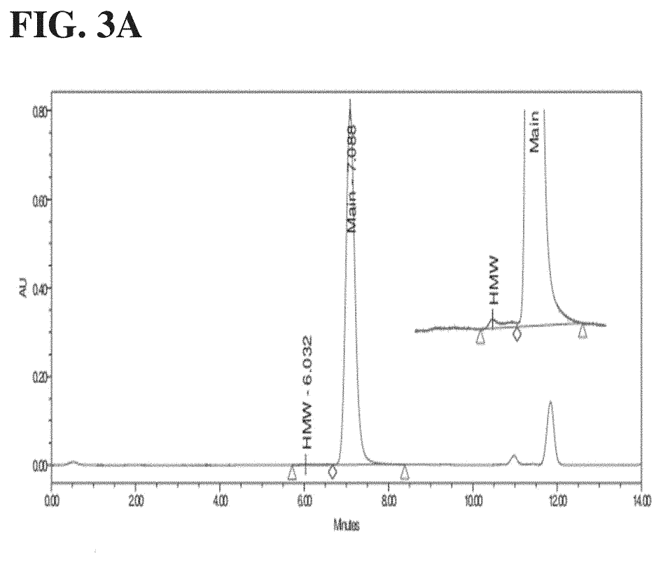

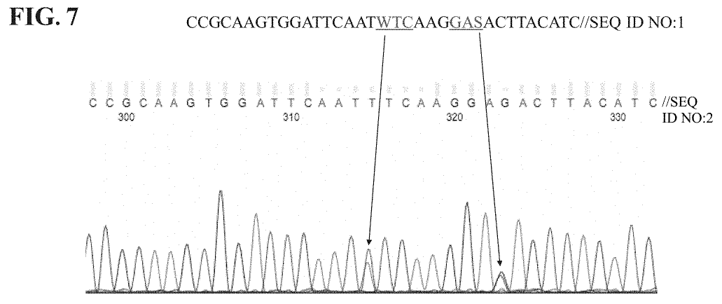

The parental sequence for mAb_A was obtained from Drugbank (accession number DB00072); this published sequence contains a proline insertion at position 217 (Eu numbering), in the heavy chain hinge region; P217 was removed for the work described herein. The parental sequence (minus afore-mentioned P217) was cloned into pcDNA3.4 by Geneart of ThermoFisher Scientific. The mAb_A variant sequences were designed, or pre-determined, by considering sites in the parental sequence liable for isomerization, deamidation, N-link glycosylation, methionine oxidation and other potential destabilizing residues for manufacture, storage and bioactivity. Free cysteines in the parental sequence were also considered as potentially detrimental to manufacturing, storage, and bioactivity. (Buchanan, A. et al., Engineering a therapeutic IgG molecule to address cysteinylation, aggregation and enhance thermal stability and expression, mAbs, 5(2): 255-62 (2013)). Every engineered site included the parental residue or the pre-selected substituted residue(s). Each codon was, therefore, degenerate. This amounted to >49000 variants. The pool of constructs was generated by Genscript (Piscataway, N.J.) in pcDNA3.1. The parental ligand was also synthesized and cloned into pcDNA3.1 by Genscript (Piscataway, N.J.). Sanger sequencing was performed on the variant plasmid pool of each the light and heavy chain separately to confirm mixed nucleotide bases were present at each of the intended codon positions (See, sequencing step in FIG. 2 and exemplary data in FIG. 7).

Expression of Antibodies and Harvest.

The mAb parental, mAb variants and the ligand were transiently expressed in the CHO-S system (Thermo Fisher Scientific Inc.). The parental mAb_A was expressed individually as per the manufacturer's instructions. Briefly, a total of 0.8 μg of plasmid DNA at a ratio of 1:1 light to heavy chain per mL of CHO-S culture was prepared with OPTIPRO™ SFM and ExpiFectamine™. The mixture was added to CHO-S cells at a viable cell density of 6×10⁶ cells/mL and greater than 98% viability. The cell culture was incubated overnight at 37° C., 80% humidity, 5% CO2 in a Thomson flask shaking at 130 RPM with a 19-mm orbit. The next day the culture was enhanced (ExpiCHO™ enhancer; Thermo Fisher Scientific Inc.) and fed (ExpiCHO™ feed; Thermo Fisher Scientific Inc.) and transferred to 32° C., 80% humidity, 5% CO2 shaking at 130 RPM with a 19-mm orbit. The second feed was performed on day 5 and the culture returned to 32° C. until harvest on day 11. Harvesting was accomplished via centrifugation at 2500×g for 10 minutes. The clarified supernatant was sterilized using an asymmetrical polyethersulfone (PES) 0.22-μM filter assembly (Nalgene). The filtrate was stored at 4° C. until purification the next day. The mAb_A variants and ligand were expressed in a similar fashion. Different expression combinations were performed in order to allow for varying degrees of complexity in the downstream processes. The mAb_A parental light chain was expressed in combination with the mAb_A heavy chain variant plasmid pool. The mAb_A parental heavy chain was expressed in combination with the mAb_A light chain variant plasmid pool. The mAb_A variant light and heavy chain plasmid pools were expressed together (data not shown).

Antibody Purification.

All of the antibody sterilized supernatants were purified using MabSelect SuRe™ resin (GE Healthcare Life Sciences) on an ÄKTApurifier (GE Healthcare Life Sciences). A 20 mM sodium phosphate, 150 mM NaCl, pH 7.4 buffer was used to equilibrate the resin. The antibody supernatant was then loaded at a flowrate calculated to produce 8.4 minute resin residence time. The resin was washed with 20 mM sodium phosphate, 150 mM NaCl, pH 7.4 buffer until the chromatographic baseline returned to column equilibration levels. Elution was then performed using 100 mM sodium acetate, pH 3.6, and fractions were collected. The fractions were immediately neutralized with 2 M Tris, pH 9. The fractions containing predominant absorbance at wavelength 280 nm were pooled into an Amicon 30-kDa ultrafiltration device for buffer exchange. The storage buffer (10 mM acetate, 9% (w/v) sucrose, pH 5.2) was used to remove the elution buffer by centrifugation with 10 volumes, three times in the Amicon concentrator. The purifications of the variant pools were performed in the same fashion. The material was submitted for SEC and then stored at 4° C. until the second purification stage (i.e., affinity chromatography step).

Size Exclusion Chromatography.

Size exclusion chromatography (SEC) analysis was performed on a Waters 2695 Separations Module high performance liquid chromatographic (HPLC) system with a Waters 2996 diode array UV detector. Twenty (20) μg of antibody material was injected on a Waters XBridge Protein SEC 7.8×300 3.5 u 200 A column. The mobile phase was 100 mM phosphate, 250 mM NaCl, pH 6.8, and the flowrate was 1 mL/min. The antibody material was detected at wavelength 220 nm at 1 Hz sampling rate during a 14 minute acquisition. The data analysis was performed using Waters Empower 3 Chromatography Data Software.

Ligand Purification and Conjugation to Solid Matrix.

Purification of ligand was performed using a nickel nitrolotriacetic acid Superflow agarose resin (ThermoFisher) on an AKTA Purifier (GE). The resin was equilibrated using 20 mM sodium phosphate, 300 mM NaCl, 20 mM imidazole, pH 7.4. The sterilized supernatant was loaded onto the resin at a 0.2 mL/min flowrate. The resin was washed with 20 mM sodium phosphate, 300 mM NaCl, 30 mM imidazole, pH 7.4, until baseline returned to equilibration level. The ligand was eluted from the resin with 20 mM sodium phosphate, 300 mM NaCl, 300 mM imidazole, pH 7.4. The elution buffer was removed using an Amicon 10 kDa ultrafiltration device and phosphate buffered saline (PBS) buffer for storage. A portion of this amount was buffer exchanged into 0.1 NaHCO₃, 0.5 M NaCl, pH 8.3, in preparation for coupling to CNBr-activated Sepharose®.

Ten (10) milligrams of ligand was conjugated to CNBr-activated Sepharose® resin as per manufacturer's recommendations (Sigma Aldrich C9142). 300 mg. of resin was swelled with 100 mL of 1 mM HCl for 0.5 hour. The resin was washed with 10 column volumes deionized water under flow followed by 3 column volumes (CV) of 0.1 NaHCO₃, 0.5M NaCl, pH 8.3. Ligand was added to the resin and mixed overnight at 4° C. The solution was allowed to flow through the resin and it was collected to determine ligand coupling efficiency (about 70%). 5 CV washes using NaHCO₃ were also collected. Unreacted groups were the blocked with 1 M ethanolamine, pH 8.0, for 2 hours at room temperature. Blocking buffer was washed away with two cycles of 5 CV of 0.1 NaHCO₃, 0.5 M NaCl, pH 8.3, followed by 5 CV of 0.1 M sodium acetate, 0.5 M NaCl, pH 4. The resin was then either used for antibody binding or stored in sodium azide, 1 M NaCl at 4° C.

Affinity Chromatography.

Ten (10) milligrams of mAb_A mixed variant pool was added to the ligand-Sepharose® resin and mixed with end-over-end mixing for 1 hour at room temperature. This batch of pooled mAb_A variants consisted of the parental light chain and the variant heavy chains. The resin was washed with PBS buffer, pH 7.4 and all unbound antibody material quantitated at optical density (O.D.) A280 nm. Elution of the bound mAb_A was performed with MgCl₂, pH 7.0, ranging from 100 mM to 3 M MgCl₂. The pH was then decreased to pH 3.6 and 1 M and 2 M MgCl₂ elutions were performed. Each fraction was measured for antibody at O.D. A280 nm. Fractions containing the most material were buffer exchanged into 10 mM acetate, 9% (w/v) sucrose, pH 5.2, to remove the MgCl₂ which might interfere with the PNGase F digestion.

Mass Spectrometry.

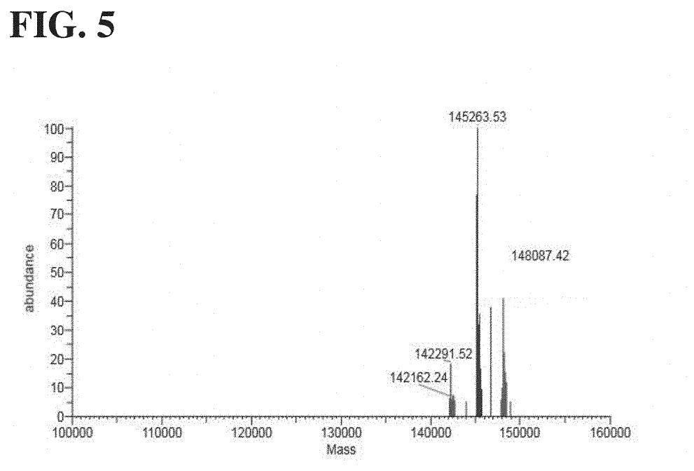

The masses of the antibodies were determined by reduced and intact mass analysis on a Thermo QE HF mass spectrometer. The samples are initially deglycosylated with Peptide:N-glycosidase (PNGase) F. The deglycosylated pool of antibodies was then split in half. One half of the pool was reduced with dithiothreitol (DTT). The other half was directly analyzed by mass spectrometer. Data for both the intact and reduced pool of antibodies was deconvoluted using the ReSpect™ deconvolution algorithm (Positive Probability Ltd.). FIG. 5 shows a representative spectrum of a deglycosylated antibody pool. The abundance of the peaks corresponds to the relative abundance of the antibody in the pool. If the separation of the eluted masses were to be insufficient, reverse phase chromatography can also used to separate the antibodies in the pool before analysis by the mass spectrometer. The deconvoluted masses were compared to the in silico (predicted or predetermined) variant molecular weights.

Water Quality.

Unless stated otherwise, water used in the production of non-commercially purchased aqueous reagents, buffers, and culture media was purified using a Milli-Q™ Synthesis A-10 water purification system (Millipore Corporation). Reagents for mass spectrometry (MS) were purchased commercially: Water LC/MS grade (product number W6-1, Fisher Scientific); water with 0.1% Formic Acid (v/v), Optima™ LC/MS Grade (product number LS118-4, Fisher Scientific); acetonitrile with 0.1% formic Acid (v/v), Optima™ LC/MS Grade (product number LS120-212, Fisher Scientific).

### Example 2. Validation of the Inventive Method

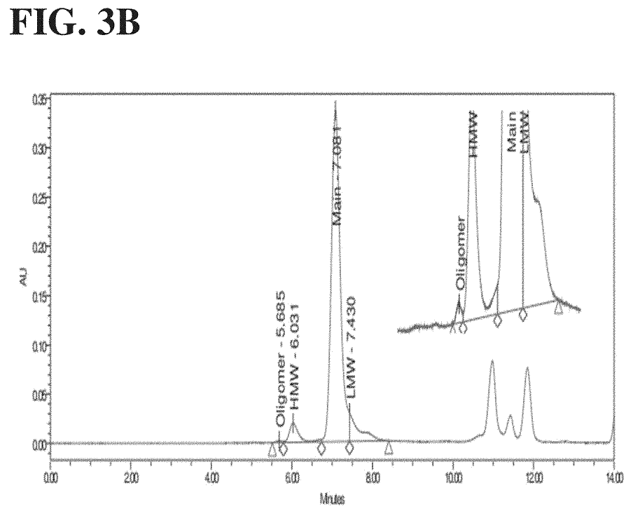

The SEC profile of the LC variants in combination with the parental HC indicated a potential issue with stability with certain LC designs. (See, FIG. 3A-C). The complexities of high molecular weight species content during the ligand affinity step were deemed too great to be given priority. This Protein A purification pool could certainly be processed further but the decision was to focus first on the lower complexity HC variants in isolation. There were 512 HC variants, when combined with the parental LC sequence. This complexity was sufficiently high to test the inventive high throughput method for screening antibody variants. The greater than 5000 different variants—when the HC variant pool and LC variant pool were combined—was too high of a complexity for initial studies along with the unpredictability of the high molecular weight species. HC variants that were found to pass the first screening could also be compared to the findings from the LC-HC variant combination pool. This analysis would allow one to determine how certain LC variants can improve the performance of HC variants that otherwise did not survive screening with parental LC. An LC variant pool can also be co-expressed with the parental HC and this can simplify the analysis. The top candidates from the HC variants alone and the LC variants alone can then be combined. But this strategy would require another round of DNA synthesis and cloning. Furthermore, this would require a complicated design strategy due to the precise codon combinations that need to be left out of the designs if library simplification is desired. The largest diversity pool would be the HC-LC variant combination and it would be the preference as a single-round screening method. The elution stringency and analysis methods decreases the complexity as the present invention is intended to do.

The mAb_A HC variants—LC parent design pool was bound to the ligand affinity column and eluted with MgCl₂ at pH 7.0 and 3.6. Antibody material began to be eluted at 500 mM MgCl₂, pH 7.0 where the peak eluted amount was at 2 M MgCl₂. Another main peak of eluted material was at 2 M MgCl₂ at pH 3.6. Approximately 40% of the total eluted material was released within the pH 7.0 range whereas about 50% was eluted within the pH 3.6 range. 10% of the material was lost in washes or flowthrough. We suspect the high concentration MgCl₂ primarily disrupted the mAb_A/ligand interaction which resulted in the release of mAb_A from the ligand affinity matrix. The lower pH indicated a further disruption of either the mAb_A/ligand interaction directly or a proximal allosteric effect where an induced conformation change on either or both proteins resulted in release of more mAb_A.

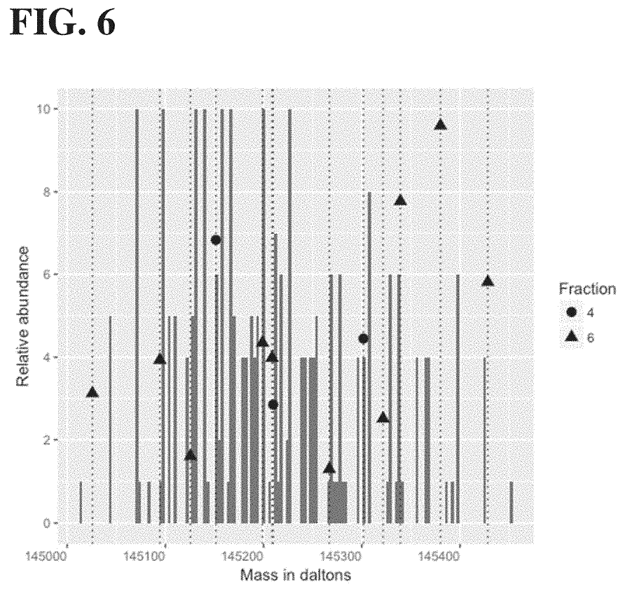

FIG. 6 shows a comparison of the calculated masses of every mAb_A HC variant with the empirically determined masses for eluted fractions 4 and 6 from the ligand affinity column. Fractions 4 and 6 were the peak eluted amounts at pH 7.0 and 3.6, respectively. The parent mAb_A empirical mass was found to be just over 1 Dalton less than the calculated mass. (145101.95 versus 145102.99, respectively). We assumed, in general, that the empirically determined HC variant masses did not deviate from the calculated masses any more than the empirically determined parental mass deviated from the calculated masses. Most of the eluted mAb_A HC variants from fraction 4 and fraction 6 were very close to calculated masses. The furthest empirical mass from any calculated mass was 145025.72. This was 11 Daltons away from the nearest calculated mass. It is unknown what the sequence of this protein could be. Although there are empirical masses that are close to calculated masses, in most cases the sequence(s) coinciding with that mass is not precisely known since there are a handful of antibodies with the same calculated mass. There were a total of 50 likely sequences that all of these empirical masses could be. However, this number of 50 possible antibodies is a magnitude less than the 512 that were designed. The relatively small number of 50 mAbs is a much more manageable number to individually produce recombinantly for further study or analysis than 512 individual variants would be. Thus, the benefit of the inventive high throughput method for selecting an antibody variant amino acid sequence of interest from a plurality of antibody variant amino acid sequences is readily apparent.

We claim:

1. A high throughput method for selecting an antibody variant amino acid sequence of interest from a plurality of antibody variant amino acid sequences, comprising the steps of:
(a) predetermining a plurality of variant amino acid sequences and the corresponding molecular weight of each member of the plurality of variant amino acid sequences, wherein the variant amino acid sequences are variants of a preselected reference antibody, wherein the parent antibody specifically binds to a target ligand of interest;
(b) cloning a plurality of nucleic acid sequences, each encoding a member of the plurality of variant amino acid sequences, to generate a mixed pool of nucleic acids capable of transfecting a mammalian cell;
(c) transfecting a plurality of mammalian cells with the mixed pool of nucleic acids from step (b);
(d) culturing the transfected mammalian cells under physiological conditions allowing the cells to express recombinant antibodies;
(e) harvesting the recombinant antibodies present in the culture in step (d) into a cell-free supernatant fraction and purifying the cell-free supernatant fraction by affinity chromatography to obtain a mixed pool of IgG molecules;
(f) loading the mixed pool of IgG molecules from step (e) onto an affinity chromatography matrix, wherein the target ligand of interest is covalently conjugated to the affinity chromatography matrix;
(g) eluting the IgG molecules from the affinity chromatography matrix under increasingly stringent buffer conditions and collecting a plurality of eluant fractions; and
(h) detecting the molecular weights of the IgG molecules present in each eluant fraction by mass spectrometry,
whereby one or more antibody variants of interest, from the eluant fraction obtained under the highest stringency buffer conditions in step (g), having a predetermined variant amino acid sequence, is identified by its corresponding molecular weight and can be selected from the plurality of variant amino acid sequences for further analysis.

2. The method of claim 1, wherein culturing the transfected mammalian cells under physiological conditions allowing the cells to express recombinant antibodies comprises conditions allowing the cells to secrete the recombinant antibodies into the culture supernatant.

3. The method of claim 1, wherein eluting the IgG molecules from the affinity chromatography matrix under increasingly stringent buffer conditions comprises employing a gradient of increasing ionic strength.

4. The method of claim 1, wherein eluting the IgG molecules from the affinity chromatography matrix under increasingly stringent buffer conditions comprises employing a pH gradient.

5. The method of claim 1, wherein eluting the IgG molecules from the affinity chromatography matrix under increasingly stringent buffer conditions comprises employing a gradient of increasing concentration of a molecule that competes for binding to the target ligand.

6. The method of claim 5, wherein the molecule that competes for binding to the target ligand is a small molecule or an oligopeptide.


SEQUENCE LISTING (2 sequences)

SEQ ID NO: 1 - DNA, 35 bp, Artificial Sequence
  Description of Artificial Sequence Synthetic oligonucleotide
ccgcaagtggattcaatwtcaaggasacttacatc

SEQ ID NO: 2 - DNA, 35 bp, Artificial Sequence
  Description of Artificial Sequence Synthetic oligonucleotide
ccgcaagtggattcaatttcaaggagacttacatc